(19) United States
(12) Patent Application Publication
(10) Pub. No.: US 2024/0385113 A1
(43) Pub. Date: Nov. 21, 2024

(54) METHOD OF DETERMINING A FUTURE COLOR VALUE OR CORRESPONDING PROPERTY AND ARRANGEMENT THEREFOR

(71) Applicant: Boehringer Ingelheim International GmbH, Ingelheim am Rhein (DE)

(72) Inventors: Jens Smiatek, Borgholzhausen (DE); Erich Bluhmki, Schwendi (DE); Oliver Burkert, Mittelbiberach (DE); Benjamin Joshua Kohler, Ravensburg (DE); Selina Link, Mietingen (DE); Melanie Miller, Erolzheim (DE)

(73) Assignee: Boehringer Ingelheim International GmbH, Ingelheim am Rhein (DE)

(21) Appl. No.: 18/686,991

(22) PCT Filed: Sep. 2, 2022
(86) PCT No.: PCT/EP2022/074465

§ 371 Date: Feb. 27, 2024

(30) Foreign Application Priority Data
Sep. 2, 2021 (EP) 21194516.7

(51) Int. Cl. G01N 21/64; C07K 1/14; C07K 16/00

(52) U.S. Cl. CPC G01N 21/6428; C07K 1/14; C07K 16/00; G01N 21/6452; G01N 2021/6439

(57) ABSTRACT

The present invention relates to determining a color value or corresponding property of a protein-containing solution or of a protein-containing product prepared therefrom, comprising exciting fluorescent radiation of the solution or product, measuring at least one property, preferably a spectrum or corresponding feature, of the fluorescent radiation, and determining, based on a correlation between the at least one property of the fluorescent radiation and the color value or corresponding property, the present or future color value or corresponding property of the solution or product.

The present invention relates to the field of color classification of protein-containing solutions or protein-containing products prepared from a protein-containing solution. In particular, the present invention relates to a method according to the preamble of claim 1 or an arrangement.

The present invention in particular relates to color classification of protein-containing solutions or products prepared therefrom where the proteins can be recombinant proteins, e.g. antibodies, monoclonal antibodies, or other therapeutic proteins. Such proteins can be produced in or by protein-producing structures, for example by prokaryotic or eukaryotic cells, in particular bacteria, fungi, yeasts, mammalian cells or another biological protein forming structure. Particularly preferably, the present invention relates to color classification of protein-containing solutions or products prepared therefrom where the proteins are monoclonal antibodies (mAbs). However, the present invention can be applied to different proteins as well.

The protein-containing solution can contain at least in one phase of the production process protein-producing structures like prokaryotic or eukaryotic cells to form the proteins contained in the protein-containing solution or fragments thereof like polysaccharides, nucleic acids, lipids, fats, membrane fragments, small molecular metabolites other host cell proteins (HCP). However, these molecules, structures or cells can be removed from the protein-containing solution before applying the present invention.

When producing proteins, the solution or product, a coloration of the protein-containing solution or product can occur. While the exact reason for the color and its intensity might be various and often cannot be determined exactly, the coloration intensity has been found to correspond to a usability of the proteins, the protein-containing solution or the protein-containing product prepared therefrom.

The higher a coloration intensity, i.e., the more intense the coloration is, the more likely the protein, the solution or the product is or becomes unsuitable for the desired application, e.g., to be used as a therapeutic agent.

Testing of drug substance and drug products regarding to its degree of coloration is a pharmacopoeia requirement according to the Ph. Eur. 8.0 Monograph 2031, 01/2012, pp. 753 to 755: “Monoclonal antibodies for human use”, in the following abbreviated Ph.Eur. Ph.Eur. requires monoclonal antibody products to be colorless to slightly colored, consequently rendering colored solutions unsuitable for its intended purpose.

Experimental studies showed that, for example, recombinant monoclonal antibodies (mAbs) as produced by Chinese hamster ovary (CHO) cells often exhibit a yellow or yellowish-brown color. Although at first glance this seems to be a minor problem, it has to be noted that the color of therapeutic drug formulations is a noticeable quality attribute with regulatory expectation, as, e.g., the Ph. Eur. Monograph 2031 requires products to be colorless or slightly colored and/or if the degree of coloration exceeds certain limitations the solution is being rejected.

The color of formulations like the solution or product can be formed by, e.g., oxidation reactions of tryptophan, glycation and Maillard reactions as well as the presence of vitamin B12. However, a molecular explanation for these effects is still under debate, which indicates that a sufficient process control by eliminating the corresponding species is problematic. Furthermore, it has to be pointed out that the magnitude of coloration increases with the protein, in particular mAbs, concentration, thereby limiting the effective dose of active pharmaceutical ingredients in the final protein-containing (drug) product.

In more detail, the Ph.Eur. introduced the so-called yellow (Y) or brown-yellow (BY) scales—among others—with subcategories between 1 and 7, whereas 7 indicates a colorless solution and 1 relates to the maximum expected color intensity, thus allowing to determine the degree of coloration of liquids.

Following a standard operating procedure, the solution is often manually compared by visual inspection to various reference solutions and thus classified either as being part of the yellow (Y) or brown-yellow (BY) color series, within the same procedure also the solutions degree of coloration is determined, as specific examples. This careful experimental standard procedure to classify the color value by human inspection is thus a time-consuming and exhausting approach. To overcome these drawbacks, sensitive and high-throughput capable method as well as more robust and reliable mapping schemes are of urgent need.

During a downstream called process for purification and/or concentrating the produced proteins in the solution and/or to produce the protein-containing product, the coloration usually cannot be removed completely. In particular, the color is either chemically part of or bound to the proteins or inseparably from the proteins otherwise. Accordingly, it often occurs that at the earliest in the very last steps of a production process it becomes apparent that the color in the end exceeds or will exceed a limit such that the whole batch of proceed protein-containing solution or product formed therefrom cannot be used at least for the desired purpose, e.g., as therapeutic agent in the desired concentration.

For classification of protein-containing solutions or products formed therefrom, by now it is unavoidable to prepare color reference solutions based on recipes defined by a regulation, and a trained expert to compare the coloration of the protein-containing solution or product with the reference color solutions in order to assign a color value, because automatic approaches to measure and evaluate a color directly, i.e. absorption spectrometry, turned out unreliable. Further, the color classification by an expert is not sensitive enough in early stages of production—where the color is still very light—to judge if certain process steps would affect the degree of coloration of the protein-containing solutions or products.

Natarajan Vijayasankaran et al: “Effect of Cell Culture Medium Components on Color of Formulated Monoclonal Antibody Drug Substance”, Biotechnol Prog, vol. 29, no. 5, 11 Jun. 2013 (2013 Jun. 11), pages 1270-1277 relates to color measurement, e.g., by means of normalized intrinsic fluorescence intensity (NIFTY) assay. For NIFTY measurement, the fluorescence of the antibody molecule was used as proxy for color as it was observed that color and fluorescence were correlated. For each antibody sample, the normalized fluorescence was determined by dividing the fluorescence peak area of the main peak by the UV absorbance peak area of the main peak, which normalizes the fluorescence response by the antibody mass contribution. The NIFTY value was subsequently determined by calculating the ratio of the normalized fluorescence of a sample to that of a monoclonal antibody reference sample.

US 2013/281355 A1 relates to a similar subject matter, namely the use of NIFTY and determining a color intensity value by calculating a ratio of normalized fluorescence of a test monoclonal antibody sample to that of a reference monoclonal antibody sample. Further, it is disclosed that NIFTY values are measured from the main peak of a size exclusion chromatogram that could be expected to remain constant through purification process if colored or uncolored protein molecules were not preferably purified.

However, during the complete process of protein production, harvesting and purification it regularly is desired to reduce the coloring. Thus, this results in NIFTY values were known to be suitable to surrogate a present coloration only.

Thus, an object of the present invention is to provide a method or an arrangement for determining a color value or corresponding property to gain control over the color of protein-containing solutions or products formed therefrom.

This object is achieved by a method according to claim 1 or an arrangement according to claim 15. Advantageous embodiments are subject to the dependent claims.

According to one aspect of the present invention, a method comprises exciting fluorescent radiation of the solution or product, measuring at least one property, preferably a spectrum or corresponding feature, of the fluorescent radiation, and determining, based on a correlation between the at least one property of the fluorescent radiation and the color value or corresponding property, the present or future color value or corresponding property of the solution or product.

That is, the present or future color value of the solution or product, or the present or future property of the solution or product, where the property corresponds to the color value, is determined. It surprisingly turned out that this can be achieved by examining the fluorescent radiation of the solution or product.

When determining the color value or corresponding property, a correlation between the at least one property of the fluorescent radiation and the color value or corresponding property is used directly or indirectly.

In particular, the spectrum of the measured fluorescent radiation or one or more features of this spectrum are used for determining the color value or corresponding property.

This can be achieved by directly or indirectly correlating the spectrum of the measured fluorescent radiation with reference fluorescent radiation spectra or correlating the one or more features of this spectrum with one or more features of reference fluorescent radiation spectra. Then, a color value or corresponding property being related to or corresponding to the reference fluorescent radiation spectrum having the maximum correlation, or being related to or corresponding to the one or more features of the reference fluorescent radiation spectrum having the maximum correlation, can be assigned to the measured fluorescent radiation, to the one or more features of this spectrum, to the solution or the product from which the measured fluorescent radiation originates.

This correlation can be performed directly i.e. by comparison of a measured spectrum with one or more reference spectra, the reference spectra having corresponding color values or corresponding properties. Then, a color value or corresponding property of the reference spectrum having the maximum correlation or satisfies a respective selection criterion concerning the correlation can be the result of the determination.

However, particularly preferably the correlation is conducted indirectly by a measure or tool considering the correlation, e.g., a regression method where regression parameters are or have been determined based on the correlation or an artificial neural network or another supervised machine learning being or have been trained based on the correlation. Particularly preferably, (advanced) multifactorial supervised regression approaches can be used, preferably trained to make use of the correlation. Thus, the measure or tool is configured to determine the color value or corresponding property based on (information concerning) the correlation between the color value or corresponding property on the one hand and the spectrum of the fluorescent radiation or one or more features of this spectrum on the other hand.

Determining in the sense of the present invention preferably means or covers using directly or indirectly information concerning the correlation between the at least one property of the measured fluorescent radiation on the one hand and the color value or corresponding property on the other hand, for assigning a color value or corresponding property to the measured fluorescent radiation, to the at least one property of the measured fluorescent radiation, to the solution from which the measured fluorescent radiation originates and/or the product from which the measured fluorescent radiation originates. In practice, the at least one property of the measured fluorescent radiation can be input to a tool, in particular software tool or computer program product, or a measure can be applied which determines a color value or corresponding property accordingly.

Protein containing solutions or products formed therefrom have been found to show fluorescence when having or tending to a yellowish or yellow-brownish coloration.

In particular when excited essentially at UV wavelengths, wavelengths of a color essentially complementary to the present or expected yellowish (Y) or brownish-yellowish (BY) color, and/or wavelengths responsible for yellow coloration, the resulting fluorescence has surprisingly found to show significant correlation such that the present or future color, in particular yellow/yellow-brownish color and/or with an intensity/spectrum can be determined/predicted.

It has been found that said fluorescence, in particular one or more properties or features of the fluorescent radiation, in particular the radiation intensity and/or maximum intensity wavelength, one or more a pairs of intensity and wavelength and/or other characteristics of the fluorescence spectrum, is/are correlated with a present or future degree/intensity of the color and/or a tendency of protein producing structures to be involved in occurrence of the color of the protein-containing solution or the protein-containing product, preferably while taking into account a protein concentration of the protein-containing solution or the protein-containing product and/or a production processing step of the production process of the protein-containing solution or the protein-containing product and/or a pH value of the protein-containing solution or the protein-containing product.

The present invention, thus preferably uses a correlation between two or more of: wavelength/magnitude pairs of excited fluorescence of the protein-containing solution or product, one or more concentrations of proteins, preferably of monoclonal antibodies (mAbs), in the solution and/or product, in particular during measurement and/or as expected (e.g., in the product), and color values (BY, Y), in particular GMP—Good Manufacturing Practice—defined color values, e.g., determined as described in European Pharmacopoeia.

According to the present invention, the above or further correlation(s) directly or indirectly are used to (automatically) determine, based on the measured excited fluorescent radiation, a present or future color value or corresponding.

It has been surprisingly found the fluorescent radiation or features thereof providing a high sensitivity and linearity of correlation with the color over a wide range of concentration of the proteins in the solution or product and/or over production process steps. Thus, even if not (yet) visible to a human eye or only visible to an extend that does not enable assessment with conventional approaches, the present invention enables assessment and/or prediction of the color of the protein-containing solution or product.

The present invention in particular makes use of high-throughput fluorescence spectra measurements and gains benefits due to automation capabilities. This can be applied in different technical contexts in synergistic manner.

By measuring the fluorescence and by correlation of the fluorescent radiation the present invention generally enables high-throughput and automatable color classification. Due to automation, for example species can be selected efficiently or progress of the production process of the protein-containing solution or product can be monitored and/or adapted closely by frequent color checks. This can facilitate avoiding too high color intensities.

Due to the high-throughput automation capabilities, the sensitivity, robustness and wide concentration range that can be determined in synergistic manner facilitates providing access to a broad application area, resulting in a method of manufacturing of recombinant proteins, enabling to track and finally control coloration of antibodies and consequently resulting in an increase in product quality by the present invention.

For this or other purposes, the method of the present invention advantageously can be applied to automatic measurement procedures using microtiter plates. Accordingly, a minimum sample volume of the protein containing solution or product is sufficient due to the high sensitivity provided. This enables close monitoring and efficient selection of protein forming structures/species.

In particular, the proposed method can successfully be applied to measure samples of protein-containing solutions in micro titer plates, e.g., in 96-well plate format which, as a result, clears the way to high-throughput automation of determining the present or future yellow/yellow-brownish color of the protein-containing solution or product.

The present invention, thus, can enable automatic identification and/or prediction of the color. The present invention preferably enables automated prediction of color values for drug substance, drug product and diluent/placebo. However, the automation capabilities can be advantageous in different context as well.

The present invention can enable identification or prediction of the color very early in the production process.

Thus, the present invention can enable identifying process parameters and conditions that influence the degree of coloration which might facilitate to enable altering processes and conditions that influence the degree of coloration, or to stop and withdraw a batch in order to avoid effort in futile cases. Consequently, the present invention can provide a particular resource saving, efficient and effective process of producing proteins in the protein containing solution and/or of producing the protein-containing product.

Alternatively or additionally, the present invention can enable selection and/or cloning of protein forming structures like (eukaryotic) cells that tend to produce desired proteins and no or a minimum color. Multiple samples of the solution can be examined with the method according to the present invention, and one or specific ones of the samples can be selected that tend to produce few color. The one or specific ones of the samples can form a breeding or cloning basis for producing proteins. This can be done alternatively or additionally to determining process parameters that enable producing less color.

The present invention can be particularly advantageously applied after an upstream process for producing proteins in the protein-containing solution, where in a downstream process the protein-containing solution is processed to clean the proteins contained in the protein-containing solution. Color is removed during the downstream process by different measures as far as possible. However, the extent to which color is removed in early downstream steps in the past was not assessed mostly due to the weak coloring at low protein concentrations. The present invention, however, facilitates determining and/or prediction of the color due to its sensitivity (value or corresponding properties) even if there is only weak coloring. Thus, the downstream process and parameters of process steps in the downstream process can advantageously be controlled or even changed in order, replaced, or removed.

The present invention can be applied to either upstream, downstream, or both. Alternatively or additionally, the present invention can be applied to formulation development, and/or product design. Again, particular high efficiency has been shown in context of processes for producing recombinant proteins, preferably antibodies, in particular monoclonal antibody (mAbs). However, the invention can be applied in different protein producing processes, in particular having a tendency to coloration like Y or BY coloration, as well.

Specifically, the prediction of color values or corresponding properties of the solution or product for increasing protein concentrations, in particular mAbs concentrations, is of favor with regard to the fact that the final protein-containing (drug) product includes high protein, in particular mAbs, concentrations. Notably, the concentration of the proteins increases significantly late in the process and crucially affects the color of the solution. A prediction of the color values at early process stages as enabled by the present invention, thus lowers the amount of trouble-shooting events at later process stages and provides reasonable estimates for control strategies and improvements in terms of lead optimization.

As a further benefit, the robust classification of color values/scales in terms of computational approaches provides a more stringent review process by regulatory agencies. Finally, the proposed approach can be implemented straightforwardly into automatized lab environments.

It has been surprisingly found that fluorescence spectra of solutions or products—even when they appear to be comparable at the first glance—exhibiting slight differences between different proteins, in particular mAbs, indicating a presence of different species that might influence the ratio of fluorescence to the degree of coloration.

One aspect of the present invention relates to use of—in particular high-dimensional and/or numerical—regression methods like machine learning approaches. Preferably, the present invention makes use of artificial neural networks (ANN) techniques for the mapping of fluorescence spectra onto the color values. Artificial neural networks in the following also are abbreviated as neural networks.

In this regard, the invention makes use of the correlation between the integrated fluorescence spectrum and the resulting color value. As an extension or instead of a (preferably one-dimensional) correlation, a multidimensional evaluation of fluorescence magnitude/wavelength pairs can be used, preferably in combination with the color values. Such multivariate numerical approach provides a significant increase of the accuracy and makes the approach applicable for all solution conditions.

With regard to the classification and prediction of color values and in particular regarding determining parameters of the regression/ANN, the present invention preferably facilitates measured fluorescence spectra to be used as input values whereas the BY or Y values corresponding be considered as the corresponding output or target values. Herewith, previous approaches which only rely on low-level classification of colors from fluorescence intensities have been improved significantly.

High correlation coefficients of, e.g., R2=0.94 with the corresponding Y and BY color scale were obtained in experiments, thereby including distinct monoclonal antibodies (mAbs), diluent solutions, improved formulations and measurement times.

The present invention has turned out to favorably be improved by correlation using (feed-forward) neural networks and/or machine learning. In the present invention the color alternatively or additionally can be determined and/or predicted by means of numerical regression and/or correlation techniques.

Besides more reliable classifications, an automated machine learning approach providing a high-throughput characterization of colored formulations which saves development time can be provided.

In a previous feasibility study, a feed-forward neural network with one hidden layer including, e.g. 48, hidden nodes and rectified linear unit activation functions for the prediction and classification of color values for drug substance, drug product and buffer solutions provided good results.

The present invention turned out particularly advantageous when machine learning or further numerical regression (classification techniques which was directly or indirectly used for the prediction) and classification of color values in the biopharmaceutical context is or are applied.

In terms of the predictive capability, the corresponding ANN preferably is trained on multivariate fluorescence intensity/protein (in particular mAbs), concentration pairs and the resulting color values, preferably as well as the information on the corresponding protein production process and/or protein concentration step.

Preferably, a non-linear mapping function between the fluorescence intensity, the protein, in particular mAbs, concentration and the color value is obtained/used.

As a prerequisite for the ANN processing, a series of low concentrated protein, in particular mAbs, solutions can be prepared and the respective fluorescence spectra are measured.

With regard to the surprisingly found (protein specific) linear relation between the fluorescence intensity and the protein, in particular mAbs, concentrations, which has been found to be valid for all relevant pharmaceutical concentrations, the resulting fluorescence intensity for the desired high protein, in particular mAbs, concentration can be computed by extrapolation.

The calculated fluorescence intensity as well as the desired protein, in particular mAbs, concentration can then be used as input values for the pre-trained ANN which predicts the resulting non-trivial color value.

In summary, a machine learning based approach for the reliable prediction and classification of color values is provided.

The proposed approach can be applied to improve protein-containing solution or product production process control. In combination with high-throughput fluorescence assays, the method paves the way towards a new control strategy for colored solutions. In addition, it may be also useful for impurity as well as outlier detection and also provides access to a refined analysis of protein, in particular mAbs, concentrations in unknown solutions.

A further aspect of the present invention relates to an arrangement comprising a fluorescence spectrometer for measuring a fluorescent radiation. A fluorescence spectrometer comprises a light source for emission of fluorescent exciting radiation and a photodetector for measuring fluorescent radiation, in particular for measuring a spectral intensity/power of the fluorescent radiation over wavelength or frequency). Further, the arrangement comprises a device adapted to carry out the method of determining the color value or corresponding property based on the measured fluorescent radiation. The described properties and advantages can apply accordingly.

A color in the sense of the present invention preferably is a characteristic of visual perception described through color categories, e.g., yellow or brown-yellow. Perception of color in the sense of the present invention derives from the stimulation of photoreceptor cells of humans (in particular cone cells in the human eye and other vertebrate eyes) by electromagnetic radiation (in the visible spectrum in the case). Color categories and physical specifications of color correspond to the wavelengths of the light that is reflected and their intensities.

A color preferably corresponds to electromagnetic radiation in wavelengths which are characteristic for stimulation of photoreceptor cells of humans causing the specific color impression. In particular, the color impression or the spectrum that results in this impression is governed by a specific light absorption property.

In particular, colors and color intensities in the sense of the present invention are specified in regulations like the European Pharmacopoeia (Ph. Eur.) 8th Edition or newer in section 2.2.2 providing reference formulations for color and intensity comparison.

Thus, a color or its intensity in the sense of the present invention is a specific color defined in the regulation if the stimulation of photoreceptor cells of humans (in particular cone cells in the human eye and other vertebrate eyes) by electromagnetic radiation result in the same neural response like the stimulation of photoreceptor cells of humans (in particular cone cells in the human eye and other vertebrate eyes) by electromagnetic radiation originating from reference formulations illuminated by a light spectrum which is essentially continuous in the visible range or as defined in the regulation.

A color value in the sense of the present invention is a specific color identifier that can be defined in a regulation and the color of an object can be determined by comparison with a reference color. The color identifier can either identify the color, the intensity or both of them. The color value does not need to be a number or specific wavelength, although this is appreciated. Thus, a color value in the sense of the present invention preferably is understood broad and covers various information to specify the color, the intensity or both of them.

The color or color value preferably corresponds to a property of the solution, the product or the proteins. Thus, a property that corresponds to the color value in particular is a suitability of the solution, the product or the proteins to be used for the intended purpose. The property in particular can be an indicator for pharmaceutical effects or side effects. The property preferably corresponds to the color value when the color value affects the property or the property depends on the color or color value, e.g. due to a regulative requirement, due to the color being an indicator for a suitability for application, or due to the color having a direct or indirect pharmaceutical effect.

A color classification in the sense of the present invention preferably specifying a color to fall within a class, i.e., category or range of colors and/or intensities of colors or color ranges. Thus, a range of similar colors or color intensities are assigned a color class or a color class is determined by a color classification process.

A color can be expressed by means of a color value or vice versa. Thus, the terms color class and color value are used interchangeably in the present invention and where the term color class is used it can be replaced with the term color value and the term color value can be replaced with the term color class. Nevertheless, the color value can define a specific color or a color range, where the specific color, however, is a color range with infinitesimal range. Preferably, the color class nevertheless covers colors and/or intensities that can be distinguished.

A protein-containing solution, herein for the sake of conciseness also merely called solution, in the sense of the present invention is a liquid that contains proteins. While in the protein containing solution proteins preferably are solved in solvent like water, the term protein-containing solution might cover suspensions where proteins are suspended, e.g., in water. Accordingly, the term protein-containing solution preferably is understood broadly while it can be specified not to cover suspensions as well if explicitly stated. The protein-containing solution preferably is a liquid used in a production process of proteins and might contain protein producing structures at least temporarily. Thus, the solution can at least temporarily be a culture for producing proteins, in particular a culture medium. However, the solution during a production process can contain the proteins without protein forming structures that can have been removed, e.g., in a downstream process for cleansing and/or concentrating the proteins.

A protein-containing product in the sense of the present invention, herein for the sake of conciseness also merely called product, is a product containing the proteins that the solution contains. However, other components of the solution preferably are removed such that the product preferably is suitable for application as a drug, for administration or otherwise is in a form for preferably direct use, in particular injection, e.g., for therapeutic purposes.

A fluorescent radiation in the sense of the present invention is radiation resulting from fluorescence effects of a substance like the solution or product containing fluorescent components. Fluorescence in the sense of the present invention preferably means the emission of light (fluorescent radiation) by the substance that has absorbed light (fluorescent exciting radiation) or other electromagnetic radiation causing luminescence, where the emitted light preferably has a longer wavelength, and therefore lower energy, than the absorbed radiation. For example, fluorescence in the sense of the present invention is when radiation is absorbed in the ultraviolet region of the electromagnetic spectrum while the emitted light is in the visible region, which might give the fluorescent substance a distinct color that can be seen or measured only when exposed to UV light.

An exciting radiation according to the present invention preferably is light directed to and absorbed by a substance having a capability to produce fluorescent radiation using the energy of the absorbed the exciting radiation. When fluorescence is excited at a specific wavelength, electromagnetic radiation of this wavelength is applied to the substance in order to cause the fluorescence, and, thus, to excite the substance to produce fluorescent radiation.

Fluorescent radiation, accordingly, is radiation produced by a fluorescent substance when exposing the substance to exciting radiation the substance herein is the solution or product.

A wavelength in the sense of the present invention preferably is the wavelength of (an intensity maximum) of electromagnetic radiation/an electromagnetic wave, in particular in the light range. Alternatively or additionally, the term “wavelength” can be used alternatively or replaced by the corresponding frequency.

An intensity in the sense of the present invention preferably is a gauge for measuring or representing the spectral power of light, preferably at the respective wavelength. While the term “intensity” primarily is used in the following, it corresponds to and can be replaced with the terms power (of the electromagnetic wave), magnitude (of the electromagnetic radiation/at the wavelength).

A property or feature of the fluorescent radiation particular preferably is one or more or multiple of a spectrum or a feature of the spectrum like an intensity and/or wavelength and/or intensity-wavelength pair, an intensity maximum, the wavelength and/or the intensity of the intensity maximum, of side maxima, properties like presence, shape and/or intensity of a shoulder besides a spectral maximum, an integral of the spectrum of fluorescent radiation, i.e., an area under the curve, or other related or deducted properties of the fluorescent radiation.

A wavelength maximum or maximum of a spectrum in the sense of the present invention preferably is an intensity or power maximum at a particular wavelength. The maximum preferably is an absolute maximum of the intensity or power in the spectrum or in a section of the spectrum. The maximum at least is a local intensity/power maximum while one or more other maxima can be contained in the spectrum. However, a maximum preferably is a peak significantly exceeding a noise level, and/or having an intensity/power exceeding a threshold which is such that merely less than 8, preferably less than 5, in particular four or less peaks of the spectrum are regarded as maxima. Accordingly, in particular variations that are close to the noise level are not regarded as maxima, but peaks that have a power with multiple the power/intensity of noise level. Preferably merely the absolute maximum within a sub-range of the spectrum is regarded as a maximum at a particular wavelength, the sub-ranges preferably are infrared (IR), in particular near infrared (NIR), the visible wavelength range (VIS) or a sub-range referred to a particular color visible to the human eye, and/or the ultraviolet range (UV) or a subrange of UVA, UVB, and/or UVC.

Further aspects of the present invention can be obtained from the claims and the following description of preferred embodiments referring to the Figures.

In the Figures:

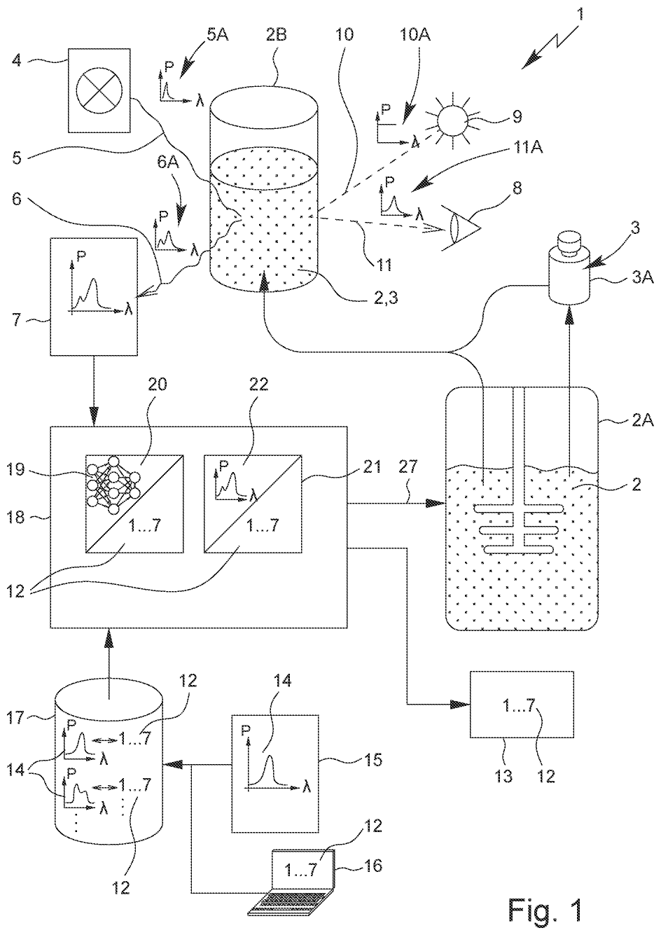

FIG. 1 shows a schematic arrangement according to the present invention;

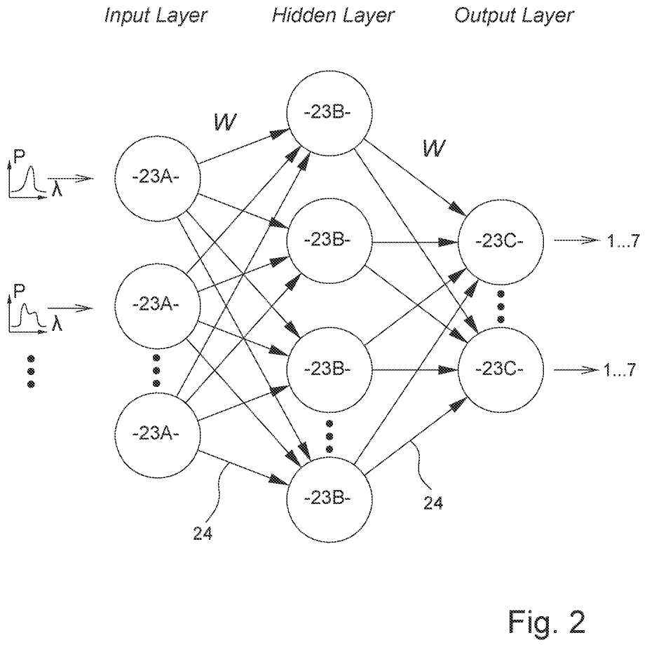

FIG. 2 shows a simplified example of an artificial neural network;

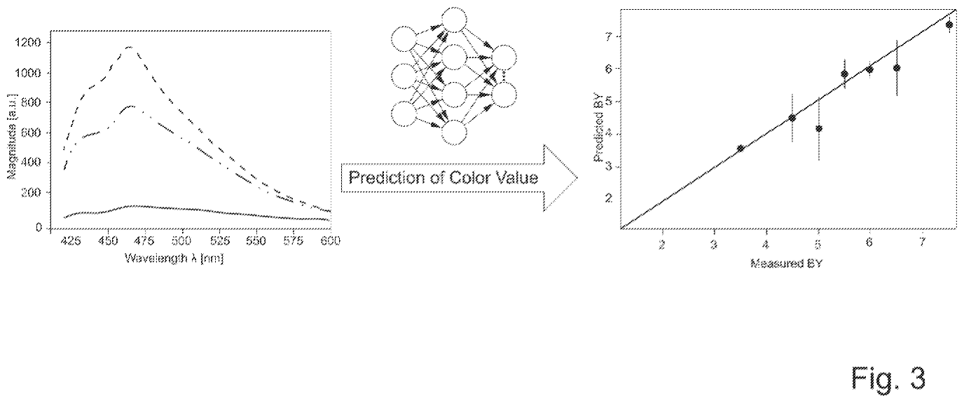

FIG. 3 shows a schematic flow chart for predicting of a color value;

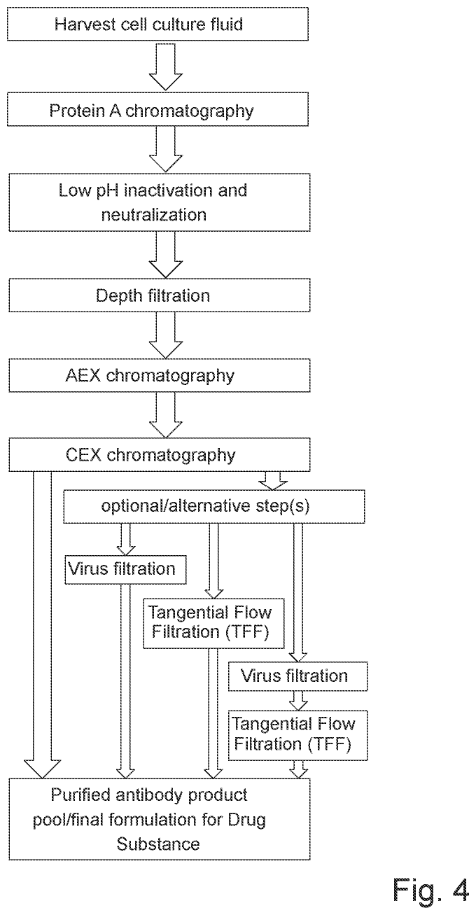

FIG. 4 shows a schematic flow chart for a downstream process;

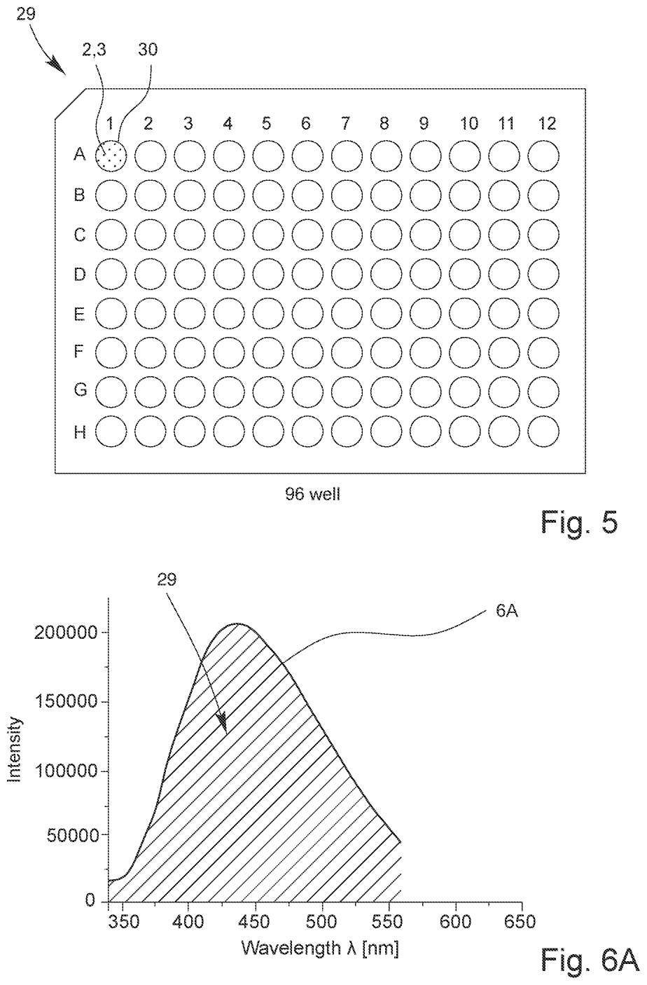

FIG. 5 shows a microtiter plate;

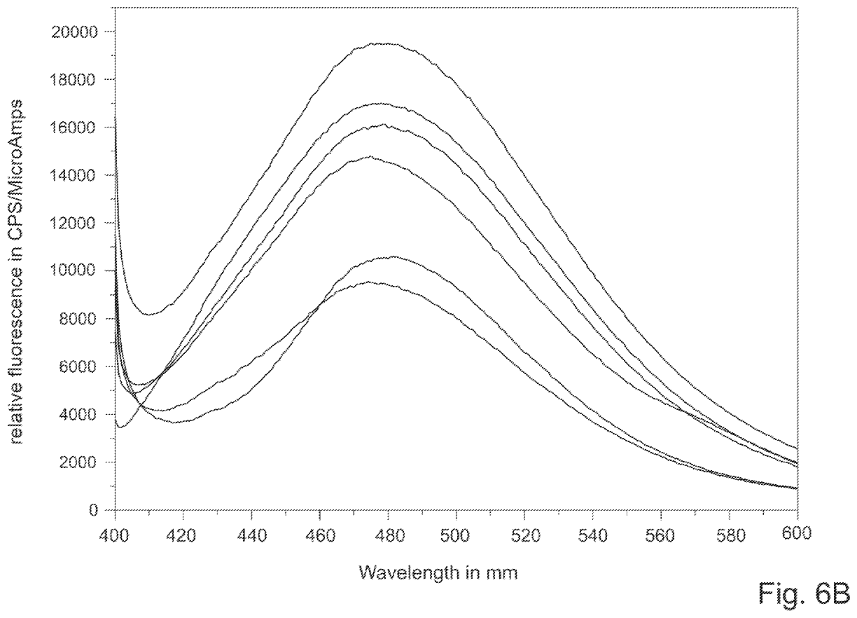

FIG. 6A shows an intensity over wavelength chart;

FIG. 6B shows an intensity over wavelength chart;

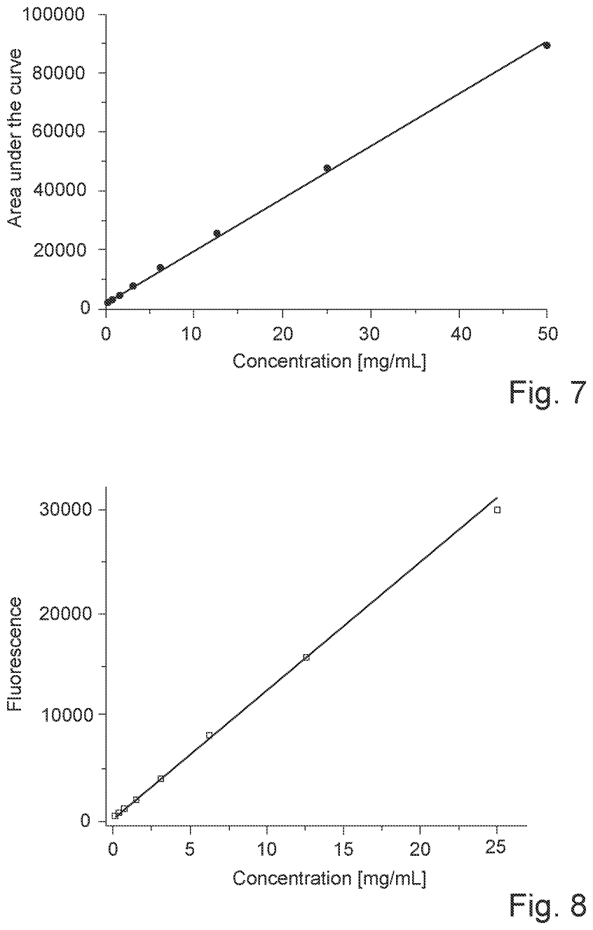

FIG. 7 shows an area under the curve over concentration chart; and

FIG. 8 shows a fluorescence over concentration chart;

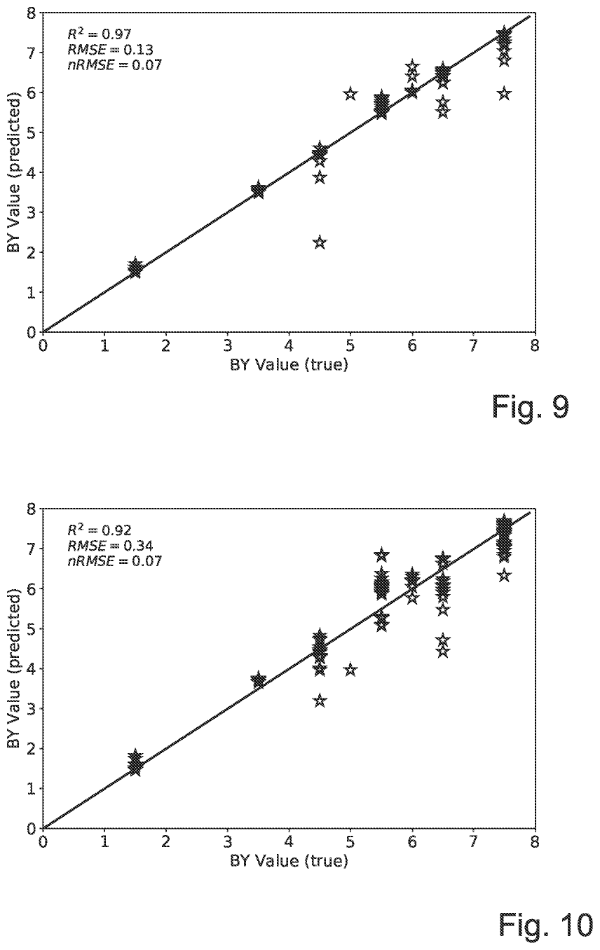

FIG. 9 shows in a chart the goodness of fit plots between predicted and measured BY values with regard to an ET model;

FIG. 10 shows in a chart the goodness of fit plots between predicted and measured BY values with regard to an ANN model;

FIG. 11 shows scaling factors p_{X} and standard deviations of scaling factors scaling factors σ(p_{X}) with values from Table 1 for the calculation of integrated fluorescence intensities;

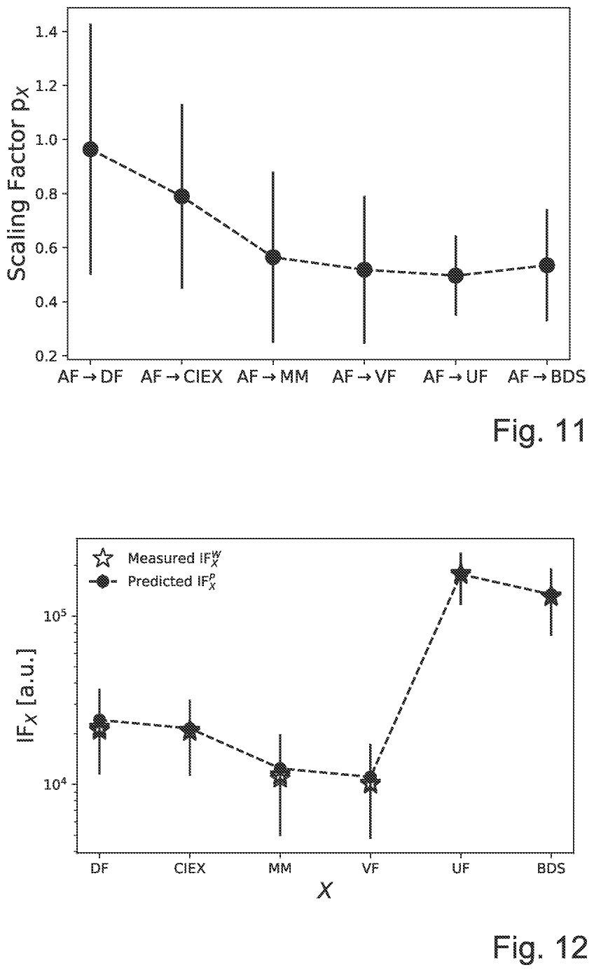

FIG. 12 shows measured and predicted fluorescence intensities with prediction errors for a chosen mAb1 at different process steps X and at given concentrations. The connecting solid grey line is just a guide for the eye;

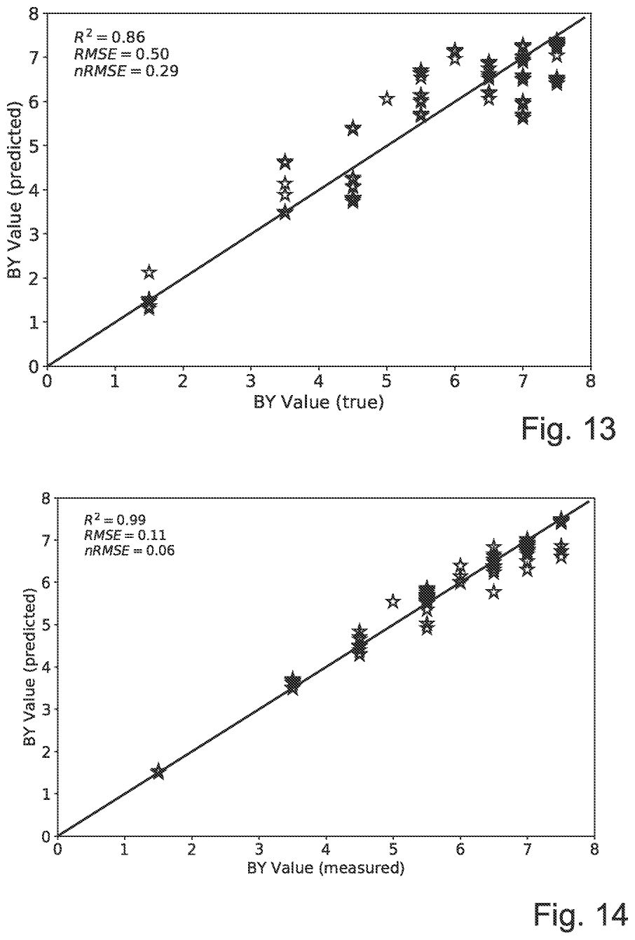

FIG. 13 shows in a chart the goodness of fit plot for predicted and measured BY values for the RF model while the straight black line shows full coincidence with a slope of 1;

FIG. 14 shows in a chart the goodness of fit plot for predicted and measured BY values for the ANN model while the straight black line shows full coincidence with a slope of 1;

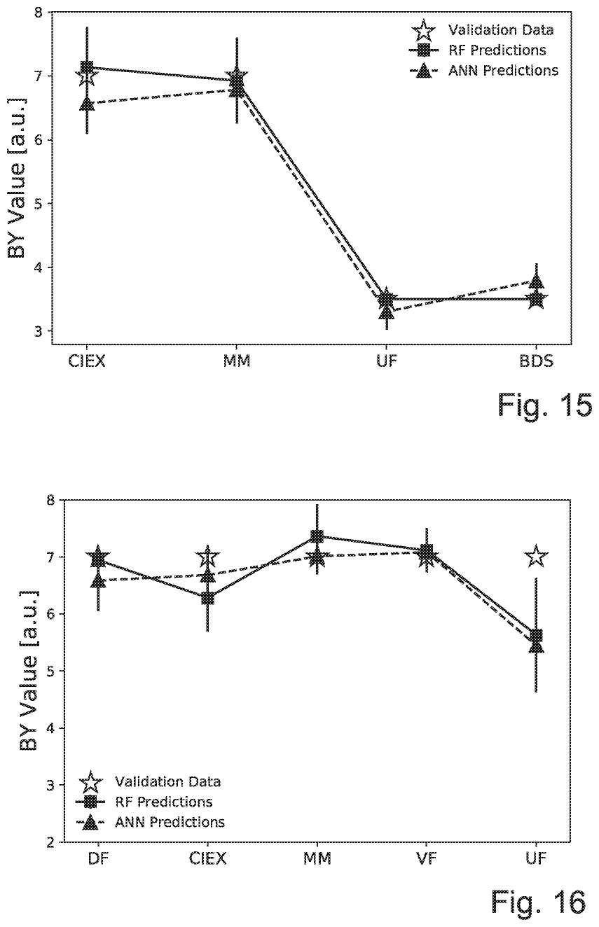

FIG. 15 shows in a chart measured BY values for certain process steps and concentrations for a specific mAb1 in combination with predicted BY values from the ANN and the ET model, where solid and dashed lines are just guides for the eyes; and

FIG. 16 shows in a chart measured BY value for certain process steps and concentrations for a specific mAb2 in combination with predicted BY values from the ANN and the RF model, where solid and dashed lines are just guides for the eyes.

In the following description of the preferred embodiments, the same reference signs are used for the same or similar parts where the same or similar effects and advantages can be achieved even if a repeated description is avoided.

FIG. 1 shows a schematic arrangement 1 according to the present invention for determining a color value 12 or corresponding property of a protein-containing solution 2, in the following abbreviated as “solution 2”, or a protein-containing product 3 prepared therefrom, in the following abbreviated as “product 3”.

In the example shown in FIG. 1, the solution 2 is contained in a bioreactor 2A and an arrow shows that optionally with further processing steps in-between the product 3 can be produced, where the product 3 is symbolized by means of a vial 3A.

Either the solution 2 or the product 3 can be examined as proposed directly or by taking a sample and examining the solution 2 or product 3 sample e.g., in a sample chamber 2B.

The proteins of the solution 2 can be produced by protein-producing structures, in particular a cell culture which can comprise, e.g., eukaryotic cells. The protein-producing structures preferably produce recombinant proteins, in particular antibodies like monoclonal antibodies (mAbs). However, the invention can be applied to different solutions 2 as well.

The protein-containing product 3 preferably is suitable for administration, in FIG. 1 symbolized for example by a vial 3A for administration by means of injection via a not shown injection device like a syringe. Of cause, use of a vial 3A is not necessary.

The product 3 and the solution 2 preferably are liquids (at room temperature). However, it is not mandatory that the product 3 is in liquid form.

The arrangement 1 comprises a light source 4 for producing fluorescence exciting radiation 5 which can be applied to the solution 2 or product 3.

The fluorescent radiation 6 emitted by the solution 2 or product 3 can be received by a spectrometer 7 of the arrangement 1.

The pictograms which are assigned to the fluorescence excited radiation 5 and the fluorescent radiation 6, respectively, schematically show exemplary spectra 5A, 6A in diagrams of light intensity P (also referred to as power or magnitude) over wavelength λ.

As can be imagined from the pictograms regarding the fluorescence exciting radiation 5 spectrum 5A, this radiation 5 preferably has a maximum in a wavelength λ range of smaller wavelengths λ than the wavelength λ range where a maximum of the of the fluorescence radiation 6 occurs, shown in the pictogram of spectrum 6A showing an example of fluorescent radiation 6 caused by fluorescence of the solution 2 or product 3.

The solution 2 or product 3 during production of the proteins preferably develops a yellowish—Y—or yellowish-brownish—BY—coloration. In the following, in a first step a usual manual approach for color classification as depicted in FIG. 1 is explained to facilitate discussion of differences the present invention enables, and to show how reference measurements can be performed.

Manually, the color can be examined by an expert's eye 8, where an at least essentially homogenous spectrum light source 9 illuminates the solution 2 or product 3 with light 10 (cf. light spectrum diagram 10A). This spectrum 10A is partially absorbed by the solution 2 or product 3, resulting in different parts of the spectrum 10A being reflected, transmitted or scattered as light 10, typically having the yellowish or brown-yellowish color.

The yellowish or brown-yellowish color preferably corresponds to electromagnetic waves having a wavelength λ maximum in the range greater than 560 nm and/or less than 620 nm. The corresponding pictogram of the spectrum 11A of the reflected light 11 schematically shows a maximum in this range, while, of course, the spectrum 11A might vary while still corresponding to an essentially yellow or brown-yellow color.

In the manual approach, the yellowish or brownish-yellowish color of the solution 2 or product 3 is compared to that of reference solutions having yellowish or brown-yellowish color in order to assign a color value 12 to the solution 2 or product 3 by manual classification.

According to the present invention, a different approach is followed as the fluorescence exciting radiation 5 might merely be a single wavelength maximum or at least a spectrum which is not continuous. The fluorescent radiation 6 caused by fluorescence of the solution 2 or product 3, which is measured by the spectrometer 7, typically is not or at least does not need to be yellowish or brownish-yellowish.

Nevertheless, it surprisingly has turned out that this fluorescent radiation 6 is correlated which the yellowish or brownish-yellowish color of the solution 2 or product 3. This correlation, thus, is used for replacing the manual classification process to assign the color value 12 although neither the fluorescence exciting radiation 5 nor the fluorescent radiation 6 needs to be yellow or brown-yellow.

According to the present invention, the fluorescent radiation 6 of the solution 2 or product 3 is exciting, at least one property, preferably the spectrum 6A or corresponding feature, of the fluorescent radiation 6, is measured and, based on a correlation between the at least one property of the fluorescent radiation 6 and the color value 12 or corresponding property, the present or future color value 12 or corresponding property of the solution 2 or product 3 is determined.

The present or future color value 12 or corresponding property of the solution 2 or product 3 is determined using the surprisingly found correlation. While it is possible to use the fluorescent radiation 6 spectrum 6A which is or represents a property of the fluorescent radiation 6a, it is preferred to use one or more features corresponding to the spectrum 6A. Such features for example are intensity P—wavelength λ pairs (points on the spectrum 6A curve/value pairs of the spectrum 6A) or particular shapes of the spectrum 6A, intensity P of a maximum, a wavelength λ of a maximum or pairs thereof, for example. All those are features corresponding to the spectrum 6A, and individual ones or combination thereof can be used for determining the color value 12 or corresponding property of the solution 2 or product 3.

The relevance of the color value 12 has already been discussed. However, one might come to the conclusion that based on the present invention the color value 12 as such can be replaced by any indicator representing a suitability for application of the solution 2 or product 3, or another indicator for which the color is relevant. On the one hand, such indicator is the color value 12 and vice versa, on the other hand such an indicator can be determined by the present invention anyway and is covered be the property corresponding to the color value 12.

For the sake of clarity, the illuminating light source 9 and eye 8 are depicted in FIG. 1 merely in order to describe the different approach while preferably neither the continuous light source 9 nor the eye 8 form part of the arrangement 1.

The fluorescence exciting radiation 5 preferably has a maximum intensity at a wavelength λ greater than 310 nm and/or less than 540 nm. More preferably, the fluorescence exciting radiation 5 has a wavelength λ greater than 360 nm and/or less than 420 nm. Particularly preferably, the wavelength λ of the fluorescence exciting radiation 5 is greater than 380 nm and/or less than 400 nm.

It has been turned out that the fluorescence exciting radiation 5 at those wavelengths k are particularly suitable for examining the solution 2 or product 3 concerning yellowish or brownish-yellowish coloring that can develop during the process of producing the product 3 with the solution 2.

Alternatively or additionally, the fluorescent radiation 6 preferably is produced by the solution 2 or product 3 and/or is detected within a wavelength λ range of 330 nm to 800 nm. That is, the fluorescent radiation 6 spectrum 6A typically contains information suitable for later classification based thereon at least between the wavelength of 330 nm to 800 nm and, thus, the spectrometer 7 preferably is capable and configured to cover at least that wavelength λ span.

The fluorescent radiation 6 preferably has a maximum intensity excited preferably at a wavelength greater than 330 nm and/or less than 800 nm. While this corresponds to the minimum sensing range of the spectrometer 7, the fluorescent radiation 6 even more preferably has a maximum or an absolute maximum greater than 420 nm wavelength and/or less than 600 nm wavelength, in particular greater than 450 nm wavelength and less than 530 nm wavelength λ.

An intensity maximum of the fluorescent radiation 6 preferably is at a wavelength λ which is more than 50, 60 or 70 nm and/or less than 130, 120 or 110 nm above the wavelength λ at which the fluorescence exciting radiation 5 has a maximum intensity. Although this already is schematically shown in the spectra 5A, 6A in the pictograms of FIG. 1, this will be discussed in further detail later.

According to the present invention, the present or future color value 12 like that from regulative specifications or a corresponding property of a solution 2 or product 3 can be directly or indirectly predicted based on the property of the excited fluorescent radiation 6 particularly precise and reliable if the above wavelength λ ranges are met, individually and in particular in a synergistic manner when combined.

Determining the present or future color value 12 or a corresponding property of a solution 2 or product 3 preferably covers directly or indirectly prediction based on the property of the excited fluorescent radiation 6 using the correlation as discussed.

Said correlations of properties of the excited fluorescent radiation 6 and the present or future color value 12 or a corresponding property preferably is or was determined by, e.g., manual classification of solution 2 or product 3 samples. The resulting reference pairs of properties of the excited fluorescent radiation 6 on the one hand and the present or future color value 12 or a corresponding property on the other hand then can be used for direct correlation or, particularly preferred, for developing a tool or measure indirectly representing the correlation while enabling a prediction of the present or future color value 12 or a corresponding property based on the properties of the excited fluorescent radiation 6.

Both is discussed in the following, starting with a direct approach followed by advantageous advanced approaches and particularly preferred properties or features used.

For representing the correlation, references, preferably reference fluorescent radiation spectra 14 or corresponding information, preferably are assigned to color values 12 or corresponding properties. Pairs of reference fluorescent radiation spectra 14 and assigned to color values 12 or corresponding properties are also referred to as pre-classified reference spectra 14. The found correlation preferably is represented by the pre-classified reference spectra 14.

Properties of reference fluorescence spectra 14 originating from reference color samples that might be characterized by means of a reference spectrometer 15. The corresponding color value 12 or corresponding property is assigned to the reference fluorescence spectra 14, respectively. This can be achieved by manual inspection and input with an input device 16 to assign the color value 12 to the property of the reference spectrum 14. A reference database 17 can be provided storing the reference spectra 14—color value 12—pairs if desired.

Determining the color value 12 or corresponding property preferably is performed by means of a correlation device 18. The correlation device 18 can examine the measured fluorescent radiation 6, i.e. the property like the spectrum 6A or one or more features thereof, using the found correlation directly or indirectly and, preferably, determine, derive or predict a color value 12 or corresponding property that can be assigned to the solution 2 or product 3.

In a direct approach, reference fluorescence spectra 14 can be checked by correlation with a measured fluorescence spectrum 6A and one of the reference fluorescence spectra 14 can be selected which has the best correlation in order to assign the color value 12 of the best correlated reference spectrum 14 to the solution 2 or product 3 which then can be output by means of the output device 13 if desired.

That is, the determined color value 12 or corresponding property can comprise or be formed by a conventionally obtained color value 12 or corresponding property which previously was obtained by an expert's comparison of a color reference solution, e.g., prepared based on a regulatory definition.

Preferred advanced approaches are based on (numerical) regression and/or artificial intelligence and/or machine learning methods or systems based on a trained machine learning structure. Here, the correlation is represented by a tool or measure that does not necessarily demand for use of the pre-classified reference fluorescence spectra 14 once fitted/trained, preferably based on the pre-classified reference fluorescence spectra 14.

The correlation device 18 preferably comprises or is formed by an artificial intelligence module 19. The artificial intelligence module 19 is configured to predict based on the property of the fluorescent radiation 6, e.g. its spectrum 6A or features thereof, the color value 12 or the corresponding property by means of an artificial intelligence considering or representing the correlation of the property of the fluorescent radiation 6 and the color value 12 or property thereof.

The artificial intelligence module 19 preferably has a pre-trained artificial intelligence structure like an artificial neural network 20 (ANN). This is indicated by the respective pictogram of the box representing the artificial intelligence module 19 in FIG. 1.

A simplified example for a possible neural network 20 is depicted in FIG. 2. Neural networks 20 consist of multiple nodes 23 and edges 24 connecting the nodes 23. There can be multiple layers of nodes 23A, 23B, 23C. In the example depicted in FIG. 2 there are three layers, one in the middle of which is called the hidden layer while in the picture on the left there are input nodes 23A and on the right there are output nodes 23C.

The artificial intelligence module 19, particularly preferably by means of the neural network 20, is configured to assign or predict based on the measured fluorescent radiation 6, preferably spectrum 6A or features thereof, the color value 12 or corresponding property of the solution 2 or product 3. It therefor can implicitly make use of the correlation of the property of the fluorescent radiation 6 and color value 12 or corresponding property, in particular by learning or having learned the correlation based on the reference spectra 14 or their features and assigned color values 12 or corresponding properties.

Alternatively or additionally, the correlation device 18 comprises a regression module 21 for predicting based on the fluorescent radiation 6, the spectrum 6A of or based on features thereof the color value 12 or corresponding property by means of a (numerical) regression method. For that purpose, regression parameters 22—in the example shown represented by a spectrum pictogram—can be or have been determined with one or more reference spectra 14 or their features. Thus, the regression parameters 22 take into account or represent the correlation of the property of the fluorescent radiation 6 and color value 12 or corresponding property.

Consequently, prediction and classification of color values 12 can be conducted by a machine learning method, in particular by one or more (feed-forward) neural networks 20, e.g., by the artificial intelligence module 19.

It has to be noted that also different or further numerical regression or classification schemes like partial least-square regression (PLS2) or other machine learning approaches like support vector machines, random forests, etc. may be used alternatively or additionally, e.g., by the regression module 21.

Hence, the present invention does not need to be restricted to the use of ANNs 20 although they surprisingly turned out to provide a high level of accuracy in the present context.

The correlation device 18, neural network 20 or regression method can be implemented in, e.g., Python by using further open source modules like TensorFlow, Keras, PyTorch or toolkits from licensed program codes like MatLab.

The neural network 20 can be pre-trained using reference spectrum 14 (or features thereof)—color value 12 (or corresponding property) pairs. By training the neural network 20 with reference spectra 14 or one or more features thereof and the assigned (now or future) color value 12 or corresponding property of the solution 2 or product 3, the behavior of the neural network 20 becomes such that an input spectrum 6A of fluorescent radiation 6 from the solution 2 or product 3 or a feature thereof can be input to the neural network 20 resulting in an output (prediction) of the most probably present or future color value 12 or corresponding property. In particular, weights W of the neural network 20 are determined or adapted by means of training the neural network 20.

In one particularly preferred example, intensity P—wavelength λ—pairs are specified and input for a plurality of pre-classified spectra 14 while a respectively corresponding color value 12 or corresponding property of the pre-classified spectrum 14 is specified as target. Accordingly, at least one weight W of the neural network 20 is determined or adapted (trained) such that the neural network 20 outputs or is configured to output (a prediction of) the specified color value 12 or corresponding property when the respective intensity P—wavelength λ—pair or (a feature of) the spectrum 6A of fluorescent radiation 6 having it or corresponding thereto is input.

A hyperparameter optimization procedure for the neural network 20 can be performed, in particular by a cross-validation scheme and/or a backpropagation algorithm can be used for the iterative adjustment of the weights W of the neural network 20.

For training the ANN, as training data a pre-classified fluorescence spectra 6A set can be used, where it surprisingly has been found that a set that includes more than 100 and/or less than 200 spectra 6A from different solutions and/or different products is sufficient for a suitable accuracy.

Alternatively or additionally, for training, testing and/or validation of the ANN, a training to test data ratio can be chosen of more than 70/30 and/or less than 90/10, preferably of 80/20.

Alternatively or additionally, for training the ANN, corresponding magnitude P—wavelength λ pairs of the spectra 6A can be used as standard descriptors, which can be iteratively mapped in the training phase onto color values 12, preferably preclassified Y and BY values.

A random selection of pre-classified fluorescence spectra 6A as training data with multiple, preferably more than 20 or 50, in particular more than 100, repetitions can enable to estimate the mean accuracy of the method for such small data sets.

After a training phase, mean Pearson correlation coefficients around <R2>=0.92+/−0.06 were obtained as well as mean root-mean-square errors (RMSEs) of RMSE=0.49 between predicted and measured BY color values 12. The obtained results thus highlight the benefits of the invention even for small data sets.

Notably, the accuracy of the predictions will increase with larger amounts of pre-classified fluorescence spectra 6A for training purposes. The presented approach thus provides a reliable classification of the actual color value 12 for the considered solution 2 or product 3.

In particular, the invention allows to predict the color value 12 of formulations for continuously increasing protein, in particular mAbs, concentrations. Here, a linear correlation between the integrated fluorescence spectrum (fluorescence intensity P over wavelength X) of the fluorescent radiation 6 and the protein, in particular mAbs, concentration can be used as a prerequisite.

The neural network 20 can be trained on the correlation between the color values 12 (target values), the protein, in particular mAbs, concentration and the fluorescence intensity P (input values).

For determining regression parameters 22 and/or training of the neural network 20, for a plurality of samples in each case an—in particular current and/or future—protein concentration of the solution 2 or product 3 and an integral 29 of the fluorescent radiation 6 intensity P—in particular an area under the curve—of the spectrum 6A of the fluorescent radiation 6 are used as inputs. Further, a current and/or future color value 12 or corresponding property is or are specified as targets. Based thereon, at least one weight W of the neural network 20 or at least one regression parameter 22 for the regression procedure can be determined or adapted. Further, it is preferred that this is done in such a way that, when the respective protein concentration and the integral of the fluorescent radiation intensity are input, the color value 12/corresponding property is predicted.

In this context, it is further preferred that, for a plurality of (reference) samples, fluorescent radiation 6A intensity P—wavelengths k—pairs and/or a production process phase is or are used as further inputs. This has turned out to improve the reliability of the outcome.

A schematic flow chart comparison of for predicted and measured color value 12 is depicted in FIG. 3. As shown on the left, several spectra 6A of different solutions 2 or products 3 have been input to a pretrained artificial neural network 20 resulting in different predicted color values 12—in the diagram on the right Y-axis values of BY (brownish-yellowish) color values 12, while the final color of the product 3 or solution 2 are measured forming the X-axis values of the BY color values 12 in the diagram on the right in FIG. 3. This on the one hand shows that the prediction works fine based on the artificial intelligence approach, because the predicted and measured color values 12 are close to or at least in the range of identity of measured and predicted BY values shown by means of a line in said diagram.

The computed fluorescence intensity P from the linear regression fit as well as the protein, in particular mAbs, concentration of the considered solution 2 or product 3 can be used as input values for the neural network 20 which then can predict the resulting color value 12. The corresponding values for the Pearson correlation coefficient between the predicted and the experimental color values are around <R2>=0.92+/−0.05, which will be improved in the near future due to larger training data sets with mAbs concentrations spanning a wide range.

For regression with the regression module 21, instead of training a neural network 20, regression parameters 22 can be determined for performing the regression in order to correlate the spectrum 6A of the fluorescent radiation 6 to determine the (present or future) color value 12 or corresponding property of the solution 2 or product 3.

When making use of the artificial intelligence module 19, the reference spectra 14 have already been considered for training the artificial intelligence, e.g., neural network 20, and do not need to be directly used for using the correlation in order to determine the color value 12 or corresponding property.

For determining the color value 12 or corresponding property, in particular the application of the neutral network 20 or the regression, preferably a production process phase and/or a protein concentration of the solution 2 or product 3—which can be measured in addition to determine the fluorescent radiation spectrum 6A of the fluorescent radiation 6—is taken into account.

For example, in early process steps a light color can be more or less serious than a similar light coloration after purifying of proteins based on the solution 2. Accordingly, either the evaluation basis or the determined color values 12 can be assessed or corrected based on or in consideration of the production process phase and/or the protein concentration.

Neutral network 20 weights W or regression parameters 20 or the neutral network 20 or regression method can have a respective input to take into account the phase and or concentration. This is particular advantageous if a coloration tendency is evaluated or a potential future color of the solution 2 or product 3 is predicted or if the present color shall be properly assessed concerning relevance of the determined color.

Alternatively or additionally, different regression parameters or neural networks 20 can be used depending on the respective phase and/or protein concentrations. The phase and/or concentration preferably is or has been taken into account when training the respective neural network 20 or determining the regression parameters 22.

For a direct approach, there might be different pre-classified spectra 14, i.e., reference sets with reference spectra 14 or corresponding features being, respectively, assigned to color values 12 or corresponding properties for particular production process phases and/or for different protein concentrations for correlation.

Finally, there can be an adaptation or correction be applied depending on the individual production process phase or protein concentration. Here, an essentially linear relationship between fluorescence intensity P and concentration has been found, based on which an extrapolation can be performed.

The correlation can be performed or take into account the similarity of a wavelength, intensity or intensity—wavelength—pair of the fluorescent radiation 6 spectrum 6A on the one hand and the present or future color value 12 or corresponding property, of the solution 2 or product 3. That is, it is not mandatory to make use of the fluorescent radiation 6 spectrum 6A as such, but alternatively or additionally features of that spectrum like wavelength-intensity-pairs can be used for the correlation.

The measured fluorescent radiation 6 preferably directly or indirectly is correlated with a present or future color value 12 or corresponding property of the solution 2 or product 3. Particularly preferably, intensity P—wavelength λ—pairs of the spectrum 6A are used for determining the color value 12 or corresponding property.

The color value 12 preferably is determined using the correlation of the spectrum 6A of the fluorescent radiation 6 or features thereof like maximum intensity wavelengths, shapes thereof or of neighboring shoulders, site maxima or the like, the shape and/or progress of the whole spectrum 6A of the fluorescent radiation 6 with references 14.

Particularly preferably, the present invention facilitates prediction of a future color value 12, or a property of the solution 2 or product 3, e.g., regarding suitability of administration as a drug. In one aspect of the present invention, a present color or corresponding color value 12 is determined by means of the determination/correlation and can then be a basis for a prediction of a color, color value 12 or property in the later process performed with the solution 2 or to be expected for the product 3.

However, alternatively or additionally, a correlation can be performed directly between the (spectrum 6A of the) fluorescent radiation 6 and a future or final color, color value 12 or corresponding property. In particular, any extrapolation can be avoided by such direct correlation of the present spectrum 6A with a future or expected property or coloration of the solution 2 or final property or coloration of the product 3.

While the correlation can be conducted by means of regression, as already indicated it is particularly preferred to make use of artificial intelligence, in particular machine learning and/or neural networks 20.

For prediction purposes, a neural network 20 can be trained or regression parameters 22 can be determined based on present fluorescent radiation spectra 6A of reference samples, and future or final color values 12 or corresponding properties.

A series of experimental fluorescence measurements have been performed at low protein, in particular mAbs, concentrations. A standard numerical regression method like a least-square approach computes the corresponding linear slope and the offset of the correlated data sets.

The correlation has been found essentially linear, that is not to change for increasing/higher protein, in particular mAbs, concentrations. Thus, results can be extrapolated to (relevant drug) product 3 concentrations.

With regard to this point, the linear regression fit is then preferably used to compute the resulting fluorescence intensity P for the desired high protein, in particular mAbs, concentration.

In one further aspect of the present invention, the production process for producing proteins in the solution 2 or for the product 3 can be controlled based on the correlation. In particular, the production process can be controlled based on the color value 12 or corresponding property, in particular color value 12 or the property corresponding thereto, which is determined based on (the property of the) fluorescent radiation 6 which can be one or more features of the spectrum 6A, while preferably at least one process parameter of the production process, in particular for purifying step, is controlled based thereon.

With other words, the outcome of the correlation according to the present invention can be used to change parameters or complete steps within a production process and, in particular, of purifying steps in order to improve the result, i.e., to reduce the color (intensity) of the final protein-containing solution 2 or product 3 produced therefrom.

In this context, FIG. 4 shows an example of a downstream process for purifying proteins produced in a protein-containing solution 2. Before applying the method, the protein-containing solution 2 can be, e.g., a cell culture. The depicted process starts with harvesting the cell culture fluid followed by a chromatography step, a preparation step for enabling a filtering which is performed afterwards and, in the end, one or more chromatography steps and further optional steps are conducted to purify the proteins, in particular for producing the (purified antibody) product 3.

According to the present invention, parameters for controlling the respective steps can be checked or modified or some of the steps can be left out or exchanged depending on the correlation outcome. In FIG. 1, process control 27 is symbolized by an arrow.

Techniques for producing protein-containing solutions in the sense of the invention are per se known to the person skilled in the art. They are preferably produced biotechnologically by culture, preferably fermentation of appropriate prokaryotic or eukaryotic cells, in particular bacteria, fungi or mammalian cells. This means, the cultivated cells express the protein of interest in cell culture in a suitable medium and under conditions that allow growth and/or protein production/expression. In view of the feeding strategy batch culture, fed-batch culture or continuous cell culture or combinations thereof are known and chosen individually in view of the demands of the cells and the intended production protocol. Cells suitable for the production of a secreted recombinant therapeutic protein may be referred to as “host cells”. In view of the physical setting the cells can be cultivated in adherent, encapsulated or and suspended form. Suspension of cells in the respective media is often preferred.

In certain embodiments, the host cell may further comprise one or more expression cassette(s) encoding a heterologous protein, such as a therapeutic protein, for example a recombinant secreted therapeutic protein. The expression of the protein of interest then occurs in a cell comprising a DNA sequence coding for the biologic product of interest or recombinant protein, which is transcribed and translated into the protein sequence including post-translational modifications to produce the biologic product of interest or recombination protein in cell culture.

In certain embodiments, the production of such a protein of interest comprises cultivating a bacterial cell, e.g. Escherichia coli as an example for gram-negative bacteria or Bacillus subtilis as an example for gram-positive bacteria both of which have been advantageously transformed with the genetic element coding for the respective protein before. The protein of interest can then be purified from the cells (e.g. from the periplasma of gram-negative bacteria) or as a secreted protein directly from the cell culture medium.

In other embodiments, the production of such a protein of interest comprises cultivating an eukaryotic cell, e.g a fungus cell. Preferred are those of the strains Pichia (e.g. Pichia pastoris) and yeast (e.g. Saccharomyces cerevisiae), especially those that secrete the protein into the cell culture medium.

In certain embodiments, the eukaryotic host cells are animal cells like insect cells or mammalian cells like rodent cells such as hamster cells or murine cells such as murine myeloma cells, such as NS0 and Sp2/0 cells or the derivatives/progenies of any of such cell line. Such mammalian cells can be isolated cells or cell lines, preferably transformed and/or immortalized cell lines. In certain embodiments, the mammalian cells are adapted to serial passages in cell culture and do not include primary non-transformed cells or cells that are part of an organ structure. In certain embodiments, the mammalian cells are BHK21, BHK TK−, Jurkat cells, 293 cells, HeLa cells, CV-1 cells, 3T3 cells, CHO, CHO-K1, CHO-DXB11 (also referred to as CHO-DUKX or DuxB11), a CHO-S cell and CHO-DG44 cells or the derivatives/progenies of any of such cell line. In certain embodiments, the mammalian cells are CHO cells, such as CHO-DG44, CHO-K1 and BHK21, and even more preferred are CHO-DG44 and CHO-K1 cells. In certain embodiments, the mammalian cells are CHO-DG44 cells. Glutamine synthetase (GS)-deficient derivatives of the mammalian cell, particularly of the CHO-DG44 and CHO-K1 cell are also encompassed. In one embodiment, the mammalian cell is a Chinese hamster ovary (CHO) cell, for example a CHO-DG44 cell, a CHO-K1 cell, a CHO DXB11 cell, a CHO-S cell, a CHO GS deficient cell or a derivative thereof.

Appropriate techniques for producing protein-containing solutions in the sense of the invention comprise advantageously the steps of: (i) cultivating a cell expressing the protein of interest in cell culture as described above; (ii) harvesting the protein of interest from the cell culture, e.g. by centrifugation, per se known to the person skilled in the art to result in the form of a fluid comprising protein of interest and one or more impurities, buffer components or other components as disclosed above (harvested cell culture fluid; HCCF); (iii) capturing or purifying the protein of interest comprising one or more chromatography steps, e.g. affinity chromatography, anion and/or exchange chromatography of the fluid comprising protein of interest, the choice or order of which is per se known to the person skilled in the art and can be designed individually for the respectice proteins and fluids; (iv) optionally one or more further steps like virus filtration and or inactivation, concentration (e.g. by ultrafiltration), buffer exchange (e.g. by diafiltration) and (v) optionally formulating the protein of interest into a pharmaceutically acceptable formulation suitable for administration.

The steps of ultrafiltration (UF) and diafiltration (DF) can advantageously be combined like this: (a) a first ultrafiltration (UF1), followed by (b) a first diafiltration (DF1) with a buffer of high ionic strength, followed by (c) a second diafiltration (DF2) with a buffer of low ionic strength, esp. with a lower ionic strength than that of the buffer for DF1, followed by (d) a second ultrafiltration (UF2). All of these steps (a)-(d) are described in further detail in WO 2018/033482 A1.

For the purification of antibodies and antibody-like proteins for example the following purification protocol can be applied: (i) Starting with the cell culture from the fermentation (preharvest cell culture fluid; CCF) of preferably mammalian cells; (ii) harvesting by centrifugation to result in the harvested cell culture fluid (HCCF); (iii) affinity chromatography with protein A; (iv) virus inactivation (VI); (v) depth filtration (DF); (vi) anion exchange chromatography (AIEX), e.g in elute-mode; (vii) cation exchange chromatography (CIEX), e.g. in bind-elute mode; alternatively a hydrophobic interaction chromatography (HIC) or a mixed mode of both chromatographies; (viii) virus filtration (VF); (ix) ultrafiltration and/or diafiltration (UF, DF), or vice versa, preferably as disclosed above; (x) to result in a bulk drug substance (BDS), which can (xi) optionally be further processed, re-buffered and/or filled in vials or in other appropriate devices.

The present invention in particular relates to color classification of protein-containing solutions or products prepared therefrom.

Particularly preferably, therefore, the correlation is performed based on a spectrum 6A of fluorescent radiation 6 originating from the solution 2 or product 3 excited during, in advance or after or with a sample obtained in one or more of the following process steps: (i) cultivating a eukaryotic cell expressing a recombinant protein of interest in cell culture; (ii) harvesting the recombinant protein; (iii) purifying the recombinant protein; and (iv) optionally formulating the recombinant protein into a pharmaceutically acceptable formulation suitable for administration; and (v) obtaining at least one sample comprising the recombinant protein in steps (ii), (iii) and/or (iv).

The approaches used in the present invention, preferably in terms of numerical regression procedures, as well as the corresponding fluorescence measurements can be fully automatized with appropriate laboratory equipment and program codes. Human efforts are only used for the preparation and mixing of the corresponding solutions 2.

The correlation according to the present invention preferably is performed automatically based on samples of the solution 2 or product 3 uptaken in a microtiter plate 29 as depicted in FIG. 5, showing an example of a microtiter plate 29 having 96 wells 30 for taking up the solution 2 or product 3.

The light source 4 can provide fluorescence exciting radiation 5 to each of the wells 30 step by step or simultaneously, and one or multiple spectrometers 7 can measure spectra 6A of fluorescent radiation 6 originating from the solution 2 or product 3 contained in the respective wells 30. This facilitates an automatization of the characterization and/or prediction of the correlation value 12 or property corresponding thereto.

FIG. 6A shows an example of an intensity over wavelength chart, a spectrum 6A, of fluorescence radiation 6 emitted by a solution 2 or product 3 when excited at 390 nm. The light intensity of fluorescent radiation 6 typically depends on the protein concentration in the solution 2 or product 3. Thus, it is appreciated to take the concentration or corresponding process phase into account when assessing the measurement results. Particularly preferably, an integral 29—area under the curve—can be used for determining the corresponding color value 12.

FIG. 6B shows an overlay of six fluorescence intensities over wavelength charts of fluorescence radiation 6 emitted from different products 3 or solutions 2 when excited at 390 nm. Every product 3 or solution 2 has been adjusted to a protein concentration of 10 mg/mL.

FIG. 7 shows an area under the curve over—concentration chart. Accordingly, the area under the curve of the spectrum of the fluorescent radiation 6 has been found being essentially linearly related to a protein concentration present or future of the solution 2 or product 3. This can be used to interpolate or extrapolate the fluorescent radiation 6 intensity P over the concentration, and/or to predict a fluorescent radiation 6 or present or future color value 12. This preferably is considered for determining the color value 12 or corresponding property. In particular, the neural network 20, thus is trained taking into account this relation, or the regression parameters 22 are fitted or determined based thereon or in consideration thereof.

FIG. 8 shows in a diagram the essentially linear relationship of measured fluorescence and protein concentration.

In a particularly preferred embodiment, the future color value 12 or corresponding property of the solution 2 or product 3 is determined using a machine learning or regression model, the model being trained using a reference spectrum 14 or features thereof, and the corresponding present or future color value 12 or corresponding property of the solution 2 or product 3.

Particularly preferably, the future color value 12 or corresponding property of the solution 2 or product 3 is determined using an artificial neural network 20 which is trained using the reference spectrum 14, the present color value 12, and the corresponding property of the solution 2 or product 3.

The machine learning model, in particular the artificial neural network 20, can be adapted dynamically and/or continuously. Alternatively or additionally, the regression model can be dynamically and/or continuously adapted. For doing so, the property of the fluorescent radiation 6 determined based on the solution 2 during the production process (e.g., after using it for prediction) and a measured present or future color value 12 or corresponding property of the solution 2 or product 3 are used as inputs for the machine learning model, in particular the artificial neural network 20, and/or the regression model to adapt it/them.

The production process phase can be taken into account when determining or forecasting the color value 12 in that the property of the fluorescent radiation 6 (preferably spectrum 6 or a corresponding feature) is normalized and/or extrapolated based on an expected protein concentration change within the process from the step where the sample of the solution 2 is taken/the fluorescent radiation 6 or its property is determined to the future step for which the future color value 12 or its property is predicted.

An in-process step specific scaling factor can be determined preferably empirically, representing an expected (preferably concentration change independent) influence of the process on the color value 12, e.g., due to purification in future process steps.

The scaling factor can be specific for the process step in which a sample of the solution 2 is taken and analyzed to predict the future color value 12 or its corresponding property 12. The scaling factor can be applied to, particularly multiplied with, the property of the fluorescent radiation 6 (preferably spectrum or a corresponding feature) or its normalized or extrapolated value directly or (indirectly) as an input variable in the prediction process to determine the future color value 12 or its corresponding property.

It is particularly preferred and/or the production process phase can be taken into account in that the property of the fluorescent radiation 6 (preferably spectrum or a corresponding feature) is: divided by a protein concentration of the protein in the protein-containing solution 2 (of the taken sample), and is multiplied with a desired bulk drug substance protein concentration (protein concentration of the protein in the desired future protein-containing solution 2 or product 3), and preferably is processed (multiplied) with the in-process step specific scaling factor.

The result is used as an input parameter for at least one of a regression procedure, preferably advanced multifactorial regression, or artificial intelligence method, preferably a supervised machine learning method, in particular for the artificial neural network 20, to finally predict the degree of coloration—i.e., the color value 12 or the corresponding property—of the bulk drug substance (product 3).

Alternatively, one or more of the steps can be implemented in the regression procedure, preferably advanced multifactorial regression, or artificial intelligence method, preferably a supervised machine learning method, in particular for the artificial neural network 20. Then, the respective parameter “protein concentration”, “desired bulk drug substance protein concentration” and/or “in-process step specific scaling factor” is/are input instead of the corresponding result.

Thus, the production process phase can be taken into account completely or partially by the regression procedure, preferably advanced multifactorial regression, or artificial intelligence method, preferably a supervised machine learning method, in particular for the artificial neural network 20.

In one option, an intermediate result of one or more of the steps and parameters for the other steps selected from “protein concentration of the protein in the protein-containing solution 2”, “desired bulk drug substance protein concentration”, and “optionally specific scaling factor” are used as input parameters.

In another option, the fluorescent radiation 6 property and the parameters “protein concentration of the protein in the protein-containing solution 2”, “desired bulk drug substance protein concentration”, and “optionally specific scaling factor” are used as input parameters.

The regression procedure, preferably advanced multifactorial regression, or artificial intelligence method, preferably a supervised machine learning method, in particular for the artificial neural network 20, thus, preferably is/are trained with respectively corresponding present of future color values 12 or corresponding properties and one or more of the following parameter (sets) of the following alternative input parameter groups A to D:
A: fluorescent radiation 6 property, protein concentration of the protein in the protein-containing solution 2, desired bulk drug substance protein concentration, optional: in-process step specific scaling factor;
B: protein-mass specific/extrapolated fluorescent radiation 6 property, desired bulk drug substance protein concentration, optional: in-process step specific scaling factor;
C: predicted specific/extrapolated bulk drug substance fluorescent radiation 6 property; desired bulk drug substance protein concentration;
D: predicted specific/extrapolated bulk drug substance fluorescent radiation 6 property multiplied with the desired bulk drug substance protein concentration.

The following is an exemplary description of the procedure to predict the coloration (represented by the color value 12 or corresponding property) of a bulk drug substance (product 3) by measuring an in-process sample (of the protein containing solution 2) of any process step:

The degree of coloration—represented by the color value 12 or corresponding property—of a bulk drug substance (product 3) predicted by exciting fluorescent radiation 6 at, e.g., 390 nm of the solution 2 derived from samples of in-process steps (of production or purification of the proteins within the protein-containing solution 2), measuring at least one property, preferably a spectrum 6A or corresponding feature (like an intensity or, e.g., an integral 29/area under the curve thereof) of the—preferably blank subtracted—fluorescent radiation 6, e.g., between 420 nm and 600 nm, which is further divided by a protein concentration of the protein-containing solution 2 sample, resulting in a protein-mass specific/normalized fluorescence intensity. The “fluorescence intensity” here and in the following is an example for the property of the fluorescence radiation 6 and can be replaced with “property fluorescent radiation 6.

Subsequently, this protein-mass specific/normalized fluorescence intensity is processed with an in-process step specific scaling factor resulting in the predicted/extrapolated bulk drug substance specific fluorescence intensity.

In a next step, the predicted bulk drug substance specific fluorescence intensity is multiplied with the desired bulk drug substance protein concentration, the result of which is used as input parameter for the advanced multifactorial regression or supervised machine learning method to finally predict the degree of coloration (color value 12 or corresponding property) of the bulk drug substance (product 3).

Consequently, this high-throughput capable procedure, in conjunction with the method's sensitivity, robustness and wide concentration range provides access to a broad application area, resulting in a method of manufacturing of recombinant proteins, that unlocks prediction, tracking and finally controlling the degree of coloration of the product 3/bulk drug substance and finally results in an increase in product quality and reduced time of process development and costs of manufacturing.

The result of a proof of concept in the following is discussed referring to FIG. 9 to 16.

In the proof-of-concept a protein concentration of 100 mg/mL for bulk drug substance (BDS) and 24 mg/mL for a protein-containing solution 2 after ultrafiltration (UF) was selected to compare the predicted degree of coloration (color value 12 or corresponding property) of bulk drug substance (product 3) of monoclonal antibody 1 (mAb1; BDS) selected as an exemplary monoclonal antibody preparation of the molecule type IgG1 at the stage of BDS and mAb2 (UF) as an exemplary respective monoclonal antibody preparation of the molecule type ZweiMab+ (as disclosed e.g. in WO 2019/234220 A1) with the measured degree of coloration according to Ph.Eur., see FIG. 15 (mAb1) and FIG. 16 (mAb2).

Classification of BY Values from Fluorescence Spectra

In the following, the application of machine learning methods for the determination of BY values (example for and in the following can be replaced with “color values 12”) from fluorescence spectra (6A) is discussed. The method is preferred supported, but not limited to certain machine learning methods, such that any advanced multifactorial supervised regression approach can be used.

### Training and Validation Data

As training and validation data for the machine learning models, it has been used a set of 123 spectra (6A) for different monoclonal antibodies (mAbs) in buffer solution (solution 2). The concentrations of the mAbs varied between 0.00 mg/mL and 84.20 mg/mL. The corresponding solutions 2 were classified by human inspection in terms of their corresponding BY values. The observed BY values were in a range between BY=7.5 and BY=1.5. In more detail, human classification introduces only integer values between BY=0 and BY=7. For intermediate results, they have been classified as “smaller than X”, meaning BY<X. This classification corresponds to the odd definition X>X+1. As Boolean logic is hard to implement into supervised machine learning regression approaches, all classifications BY<X as X.5 for training and prediction purposes were encoded.

### Computational Details

All source code was written in Python 3.7.4 [1] with the modules NumPy 1.16.5 [2] and Scikit-Learn 0.21.3 [3]. The artificial neural network 20 (ANN), random forest (RF) and extra trees (ET) models with the results shown in FIGS. 9, 10, 13, 14 and 15 were implemented using the functions MLPRegressor, RandormForestRegressor and ExtraTreesRegressor as part of Scikit-Learn [3]. If not otherwise mentioned, hyperparameters were set to default values. For the ANN model, a MinMax scaler as implemented in the function MinMaxScaler as part of Scikit-Learn has been used.

### Training and Validation of Machine Learning Models

The corresponding fluorescence spectra 6A S(W) were all monitored with a step size of 2 nm for wave lengths between Wₛ=420 nm and Wₛ=600 nm. For training purposes and later application of the machine learning models, the fluorescence spectra 6A as input values were used, where the individual values of the magnitude for a given wavelength M(W) were used as feature values and the corresponding wavelengths as features or descriptors. As target values, the corresponding by human inspection pre-classified BY values (property or spectra 6A) were used.

Different machine learning models including artificial neural networks 20 (ANN), random forest (RF), extra trees (ET) or gradient boosting among others have been trained and validated in terms of a leave-one-out cross-validation (LOOCV) approach [4]. This means that N−1 data points were used for training and the remaining data point was used for validation. Random permutation of training data and the LOOCV approach finally allowed to evaluate the statistical predictive accuracy of the individual models. As standard statistical values, the root-mean-squared error of prediction was used,

$$RMSE = \sqrt{\frac{{\sum }_{i}^{N} {( B {Y}_{i}^{P} - B {Y}_{i}^{M} )}^{2}}{N}}$$

where BYᵢ^{P} and BYᵢ^{M} denote the corresponding predicted (index P) and measured BY values (index M), respectively, for a specific data point i in the data set for N entries. The normalized RMSE (nRMSE) is defined as nRMSE=RMSE/σ(BY), where σ(BY) represents the standard deviation of BY values across the whole data set. Moreover, the Pearson correlation coefficient R² for predicted and measured BY values were calculated.

### Results

FIGS. 9 and 10 show the goodness of fit plots for validation data with regard to the LOOCV approach between predicted and measured BY values for an ET (FIG. 9) and an ANN model (FIG. 10). The ET model was trained with 100 estimators. The ANN model included three hidden layers with 30, 20 and 10 nodes. A logarithmic activation function was used. The corresponding results show a reasonable accuracy of the model predictions. The ET model performs slightly better when compared to the ANN model with RMSE values of 0.13 (ET) and 0.33 (ANN) in this example. As already discussed in section 1.1, BY values are classified in terms of integer values. In terms of the outcome RMSE<<1 for all model predictions, it can be concluded that the prediction and classification accuracy fully meet the individual purposes. It has to be noted that larger data sets with a more equally distributed amount of BY values may increase the accuracy of predictions.

### Prediction of Color Values 12 for Future Process Steps and Concentrations

In the following section, an approach for the prediction of color values 12 and integrated fluorescence intensities after certain process steps and for chosen mAbs concentrations is described. As process steps, it have been considered protein-containing solutions 2 (also to be regarded as in-process probes) each resulting after the respective following process steps of per se well-known protein production by fed-batch cultivation of Chinese hamster ovary (CHO) cells expressing transgenes coding for the respective protein and secreting the same into the cell culture medium from which the protein is purified in various consecutive steps thereafter each resulting in the respectively more and more purified protein-containing solution 2:

Affinity chromatography (AF), depth filtration (DF), cation-exchange chromatography (CIEX), mixed-mode chromatography (MM), virus filtration (VF), ultrafiltration (UF) and/or diafiltration and pure bulk drug substance in buffer solution (BDS). Further process steps like anion-exchange chromatography or liquid-liquid separation can easily be integrated into the framework.

### Scaling Factors

The measured fluorescence spectra 6A S_{X}(W) for a chosen process step Xϵ[AF, DF, CIEX, MM, VF, UF, BDS] for different wavelengths W between the chosen initial wavelength Wₛ and the final wavelength Wₑ are integrated according to

IF_{X}=∫_{W}_{ₛ}^{W}^{ₑ}S_{X}(W)dW

which yields the fluorescence intensity IF_{X} as a scalar value after using a standard discrete trapezoidal integration routine (function np.trapz( ) in Numpy [4]). It has been noticed a linear correlation between the fluorescence intensities and the considered concentrations for a chosen mAbs leading to the relation IF_{X}˜m_{X}^{Ab}c_{X} with the constant slope m_{X}^{Ab}. For the following prediction of fluorescence intensities for a chosen concentration and process step, it has been made use of this relation in terms of introducing a reference slope. The reference slope is defined by the corresponding value of the fluorescence intensity at a given concentration for protein affinity chromatography (AF). In consequence, all further predictions (extrapolations) of process (fluorescent radiation 6) intensities are related to this reference slope, such that for forthcoming projects only the fluorescence intensity and the concentration of the mAb for AF needs to be measured. Hence, one obtains constant scaling factors p_{X}=m_{X}^{Ab}/m_{AF}^{Ab} for each individual process step. In more detail, the scaling factors are calculated by

$${p}_{X} = \frac{{N}_{A F}}{{N}_{X}} \frac{{\sum }_{i}^{{N}_{X}} {\sum }_{j}^{{N}_{A F}} {IF}_{X , i} {c}_{AF , j}}{{\sum }_{i}^{{N}_{X}} {\sum }_{j}^{{N}_{A F}} {IF}_{A F , j} {c}_{X , i}}$$

where N_{AF} and N_{X} denote the number of available fluorescence intensities with the running indices i,j at the corresponding measured concentrations c_{AF,j} and c_{X,i}. The fluorescence intensities and concentrations of different standard mAbs, ZweimAbs and double mAbs in combination with the mAbs concentrations c_{X} at the corresponding process stage have been used. In total, 78 spectra 6A over all process steps with concentrations ranging from 2.4 mg/mL to 143.15 mg/mL have been considered. The corresponding values of the scaling factors are presented in Table 1 and in FIG. 9 and FIG. 10.

TABLE 1
Scaling factors p_{X} and standard deviations of scaling factors
σ(pX) for the calculation of integrated fluorescence
intensities for different process steps with regard to
the reference slope for the first process step AF.

| Process Step | Scaling factor p_{X} | Standard deviation σ(p_{X}) |
|---|---|---|
| AF → DF | 0.96 | 0.46 |
| AF → CIEX | 0.79 | 0.34 |
| AF → MM | 0.56 | 0.32 |
| AF → VF | 0.52 | 0.27 |
| AF → UF | 0.50 | 0.15 |
| AF → BDS | 0.53 | 0.21 |

It has to be noted that these scaling factors are universally applicable, meaning that the individual format is not relevant. Notably, one can clearly recognize the influence of the different process steps on the resulting fluorescence intensities, such that later process steps lead to lower fluorescence intensities. The corresponding predicted fluorescence intensity IF_{X}^{P} can now be estimated in terms of

$${IF}_{X}^{P} = {p}_{X} \frac{{IF}_{A X}^{M}}{{c}_{A X}^{M}} {c}_{X}^{P}$$

where IF_{AX}^{M} and c_{AX}^{M} denote the measured fluorescence intensity and concentration of the mAb for protein affinity chromatography (AF) and c_{X}^{P} the corresponding chosen concentration at a specific process step. A comparison between predicted and measured fluorescence intensities is shown in FIG. 11: Scaling factors p_{X} and standard deviations of scaling factors scaling factors σ(p_{X}) with values from Table 1 for the calculation of integrated fluorescence intensities.

### Training and Validation

For training of the machine learning models, a process step-independent dataset composed of different spectra for different mAbs has been used. The data set included 153 fluorescence intensities with minimum and maximum values of 5824.24 a.u. and 180030.66 a.u. The corresponding minimum and maximum concentrations were 0.00 mg/mL and 143.15 mg/mL with the minimum and maximum BY values of 7.5 and 1.5 as defined in section 1.1.

As machine learning approaches, an artificial neural network (ANN) and a Random Forest (RF) model were trained. The ANN consisted of one hidden layer with 100 nodes. A rectified linear unit (ReLu) activation function and a constant learning rate of 0.001 was used. The RF model was trained with 100 tree estimators. The corresponding results for the validation data as calculated by a leave-one-out approach are presented in FIG. 12: Measured and predicted fluorescence intensities with prediction errors for a chosen mAb1 at different process steps X and at given concentrations. The connecting line is just a guide for the eye. As input features, the concentration and the fluorescence intensity which were correlated with the corresponding BY values as targets has been used.

The corresponding accuracy of predictions for the experimental data set when splitted into testing and training data in accordance with a leave-one-out approach is demonstrated in FIGS. 13 and 14: Goodness of fit plot for predicted and measured BY values. FIG. 13: Results for the RF model. FIG. 14: Results for the ANN model. The straight black line shows full coincidence with a slope of 1. As can be seen, the RF model performs slightly better when compared to the ANN model in terms of lower normalized root-mean-squared errors (nRMSE) of predictions. It becomes clear that various regression-based machine learning approaches are able to provide reasonable prediction accuracies.

### Prediction of BY Values for Future Process Steps

The corresponding predictions of the ET model and the ANN model for two mAbs, one IgG1 (mAb1) and one Zweimab+ (mAb2) with given concentrations for different process steps are shown in FIG. 15: Measured BY values for certain process steps and concentrations for a specific mAb1 in combination with predicted BY values from the ANN and the ET model; and FIG. 16: Measured BY value for certain process steps and concentrations for a specific mAb2 in combination with predicted BY values from the ANN and the RF model. Solid and dashed lines are just guides for the eyes. As can be seen, the predicted BY values coincide with the measured BY values for validation purposes. Moreover, it can be seen that the differences between the RF and the ANN model predictions are rather marginal.

Hence, it can be concluded that the proposed approach including scaling factors in combination with machine learning provides a reliable prediction of BY values for future process steps and arbitrarily chosen concentrations. As a prerequisite, only the concentration of the mAb and the corresponding fluorescence intensity for AF need to be known. All other fluorescence intensities and color values can be predicted straightforwardly after applying the scaling factors and the pre-trained machine leaning models.

### REFERENCES

[1] G. Van Rossum and F. L. Drake, Python 3 Reference Manual, CreateSpace, Scotts Valley, CA, 2009.
[2] C. R. Harris, K. J. Millman, S. J. van der Walt, R. Gommers, P. Virtanen, D. Cournapeau, E. Wieser, J. Taylor, S. Berg, N. J. Smith, R. Kern, M. Picus, S. Hoyer, M. H. van Kerkwijk, M. Brett, A. Haldane, J. F. del Rio, M. Wiebe, P. Peterson, P. Gerard-Marchant, K. Sheppard, T. Reddy, W. Weckesser, H. Abbasi, C. Gohlke and T. E. Oliphant, Nature, 2020, 585, 357-362.
[3] F. Pedregosa, G. Varoquaux, A. Gramfort, V. Michel, B. Thirion, O. Grisel, M. Blondel, P. Prettenhofer, R. Weiss, V. Dubourg, J. Vanderplas, A. Passos, D. Cournapeau, M. Brucher, M. Perrot and E. Duchesnay, J. Mach. Learn. Res., 2011, 12, 2825-2830.
[4] T.-T. Wong, Pattern Recogn., 2015, 48, 2839.

Further aspects of the invention are:

1. Method of determining a color value (12) or corresponding property of a protein-containing solution (2) or of a protein-containing product (3) prepared therefrom, characterized in exciting fluorescent radiation (6) of the solution (2) or product (3), measuring at least one property, preferably a spectrum (6A) or corresponding feature, of the fluorescent radiation (6), and determining, based on a correlation between the at least one property of the fluorescent radiation (6) and the color value (12) or corresponding property, the present or future color value (12) or corresponding property of the solution (2) or product (3).

2. Method according to aspect 1, characterized in that (a) a fluorescence exciting radiation (5) has a maximum intensity (P) at a wavelength (λ) greater than 310 nm and less than 540 nm, preferably greater than 360 nm and less than 420 nm, in particular greater than 380 nm and less than 400 nm, and/or (b) the fluorescent radiation (6) is detected within a wavelength (λ) range at least from 330 nm to 800 nm, and/or (c) the fluorescent radiation (6) has a maximum intensity (P) at a wavelength (λ) greater than 330 nm and less than 800 nm, preferably greater than 420 nm and less than 600 nm, particularly greater than 450 nm and less than 530 nm; and/or (d) the an intensity (P) maximum of the fluorescent radiation (6) is at a wavelength (λ) which is more than 60 nm and/or less than 130 nm above the wavelength (λ) at which the fluorescence exciting radiation (5) has a maximum intensity (P), and/or (e) the color value (12) or corresponding property corresponds to a yellowish or brownish-yellowish coloration, preferably characterized in that light being transmittable, reflectable or scatterable by the solution (2) or product (3) with a maximum transmittance, reflectance or scattering at a wavelength (λ) greater than 560 nm and/or less than 620 nm.

3. Method according to aspect 1 or 2, characterized in that the solution (2) or product (3) comprises recombinant proteins or antibodies, preferably monoclonal antibodies, and the color value (12) or corresponding property is a characteristic of their, preferably pharmacological, usability.

4. Method according to any one of the preceding aspects, characterized in that a production process phase is taken into account for determining the color value (12) or corresponding property, and/or that a protein concentration is taken into account for determining the color value (12) or corresponding property.

5. Method according to any one of the preceding aspects, characterized in that the fluorescent radiation (6) has a wavelength (λ) and/or intensity (P), in particular a value pair of a wavelength (λ) and a corresponding intensity (P), based on which the present or future color value (12) or corresponding property of the solution (2) or product (3) is determined.

6. Method according to any one of the preceding aspects, characterized in that the present or future color value (12) or corresponding property of the solution (2) or the product (3) is determined using of a regression procedure or artificial intelligence, preferably Machine Learning, in particular an artificial neural network (20), based on the at least one property of the fluorescent radiation (6).

7. Method according to any one of the preceding aspects, characterized in that determining the color value (12) or corresponding property is effected by means of a pre-trained neural network (20) or a regression procedure for numerical regression or classification procedure in by means of at least one regression parameter (22).

8. Method according to aspect 7, characterized in that the artificial neural network (20) is pre-trained, the at least one regression parameter (22) is determined, the method comprises pre-training of the artificial neural network (20) or the method comprises determining the at least one regression parameter (22), wherein intensity (P)—wavelength (λ) pairs of the fluorescent radiation (6), preferably taken from the fluorescent radiation spectrum (6A), are specified as input (25) for a plurality of solutions (2) or products (3), a color value (12) or corresponding property is specified as target (26), and the at least one weight (W) defining a property of the neural network (20) or the at least one regression parameter (22) is configured, or is determined, or is adapted such that the artificial neural network (20) based on the artificial neural network (20) or the regression procedure based on the regression parameter (22) outputs or is configured to output the specified color value (12) or corresponding property when the respective intensity (P)—wavelength (λ) pair is input.

9. Method according to aspect 7 or 8, characterized in that based on the at least one property of the fluorescent radiation (6), the color value (12) or corresponding property of the solution (2) or product (3) is predicted for a future phase of the production method for producing the solution (2) or product (3).

10. Method according to any one of aspects 7 to 9, characterized in that the artificial neural network (20) is pre-trained, the at least one regression parameter (22) is determined, the method comprises pre-training of the artificial neural network (20) or the method comprises determining the at least one regression parameter (22), wherein for a plurality of samples in each case (a) an—in particular current and/or future—protein concentration of the solution (2) or product (3), and/or (b) an integral of the fluorescent radiation (6) intensity (P)—in particular an area under the curve of the fluorescent radiation (6) intensity (P), are used as input, and a current and/or future color value (12) or corresponding property of the solution (2) or product (3) is specified as target, and a weight (W) defining a property of the artificial neural network (20) or the at least one regression parameter (22) for the regression procedure is or are determined or adapted such that, when the respective protein concentration and the integral of the fluorescent radiation (6) intensity (P) are input, the color value (12) or corresponding property is predicted.

11. Method according to aspect 10, characterized in that for the plurality of samples a fluorescent radiation (6) intensity (P) wavelength (λ) pair and/or a production process phase is or are used as further inputs; and/or that the fluorescent radiation (6) parameter or color value (12) or corresponding property of the solution (2) or product (3) is or are predicted.

12. Method according to any one of aspect 10 or 11, characterized in that the production process is controlled based on the fluorescent radiation (6) parameter or based on the color value (12) or corresponding property determined based on the fluorescent radiation (6), preferably wherein at least one process parameter of the production process, in particular of a purifying step, is determined or controlled based on the fluorescent radiation (6) parameter or color value (12) or corresponding property.

13. Method according to any one of the preceding aspects, wherein the fluorescent radiation (6) parameter is determined from a sample of the solution (2) or product (3) when held in a well (30) of a microtiter plate (29).

14. Method for producing a protein-containing solution (2) or a product (3) produced therefrom, comprising the steps of: (i) cultivating a eukaryotic cell expressing a recombinant protein of interest in cell culture; (ii) harvesting the recombinant protein; (iii) purifying the recombinant protein; and (iv) optionally formulating the recombinant protein into a pharmaceutically acceptable formulation suitable for administration; and (v) obtaining at least one sample comprising the recombinant protein in steps (ii), (iii) and/or (iv); wherein the sample is the solution (2) or product (3), and wherein the method further comprises performing the method steps according to any one of the preceding aspects.

15. Arrangement (1) comprising alight source (4), a spectrometer (7) for measuring a fluorescent radiation (6), and a device (18) adapted to carry out the method according to any of the preceding aspects based on the fluorescent radiation (6).

Different aspects of the present invention can be realized independently or combined, where various synergistic effects can be obtained even if not mentioned explicitly herein.

### LIST OF REFERENCE SIGNS

1. arrangement
2. protein-containing solution
2A bioreactor
2B sample chamber
3. product
3B vial
4. light source
5. fluorescence exciting radiation
5A spectrum
6. fluorescent radiation
6A spectrum
7. spectrometer
8. eye
9. continuous light source
10. illuminating light
10A continuous light spectrum
11. reflected light
11A reflection spectrum
12. color value
13. output device
14. reference spectra
15. reference spectrometer
16. input device
17. database
18. device
19. artificial intelligence module
20. neural network
21. regression module
22. regression parameter
23. node
24. edge
25. input
26. output/target
27. process control
28. integral
29. microtiter plate
30. well
AF affinity chromatography
AEX anion-exchange-chromatography
BDS bulk drug substance
CIEX cation-exchange-chromatography
DF depth filtration
MM mixed-mode-chromatography
P power/intensity/magnitude
UF ultrafiltration/diafiltration
VF virus filtration
W weight
λ wavelength

1. A method of determining a future color value (12) or corresponding future property of a protein-containing solution (2) or of a protein-containing product (3) prepared therefrom, comprising:
exciting fluorescent radiation (6) of the solution (2) or product (3),
measuring at least one property, preferably a spectrum (6A) or corresponding feature, of the fluorescent radiation (6), and
determining the future color value (12) or corresponding future property of the solution (2) or product (3) based on a correlation between the at least one property of the fluorescent radiation (6) and a present or the future color value (12) or corresponding property of the solution (2) or the product (3).

2. The method according to claim 1, wherein the present color value (12) is determined by means of the correlation and used as a basis for a prediction of the future color value (12) or corresponding future property.

3. The method according to claim 1, wherein the correlation is performed directly between the at least one property of the fluorescent radiation (6) and the future color value (12) or corresponding future property of the solution (2) or the product (3).

4. The method according to claim 1, wherein the present or future color value (12) or corresponding property of the solution (2) or the product (3) is determined based on the at least one property of the fluorescent radiation (6) by using an artificial intelligence, preferably a Machine Learning model, in particular an artificial neural network (20), or using a regression model, preferably for numerical regression or classification by means of at least one regression parameter (22), wherein the artificial intelligence, preferably the Machine Learning model, in particular the artificial neural network (20), is trained or the regression model is determined using a reference spectrum (14) or features thereof, and the present or future color value (12) or property of the solution (2) or the product (3) corresponding to the present or future color value (12).

5. The method according to claim 4, wherein the artificial intelligence, preferably the Machine Learning model, in particular the artificial neural network (20), is trained or the regression model is determined, wherein intensity (P)—wavelength (λ) pairs of the fluorescent radiation (6), preferably taken from a fluorescent radiation spectrum (6A), are specified as input (25) for a plurality of solutions (2) or products (3), a present or future color value (12) or corresponding property is specified as target (26), and the at least one weight (W) defining a property of the neural network (20) is configured, or is determined, or is adapted such that the artificial neural network (20) based on the artificial neural network (20) outputs or is configured to output the specified present or future color value (12) or corresponding property when the respective intensity (P)—wavelength (λ) pair is input.

6. The method according to claim 4, wherein based on the at least one property of the fluorescent radiation (6), the future color value (12) or corresponding future property of the solution (2) or product (3) is predicted for a future phase of the production method for producing the solution (2) or product (3).

7. The method according to claim 4, wherein the artificial neural network (20) is pre-trained or the method comprises pre-training of the artificial neural network (20), wherein for a plurality of samples in each case
a. an—in particular current and/or future—protein concentration of the solution (2) or product (3), and/or
b. an integral of the fluorescent radiation (6) intensity (P)—in particular an area under the curve of the fluorescent radiation (6) intensity (P)
are used as input, and a present and/or future color value (12) or corresponding property of the solution (2) or product (3) is specified as target, and a weight (W) defining a property of the artificial neural network (20) is determined or adapted such that, when the respective protein concentration and/or the integral of the fluorescent radiation (6) intensity (P) are input, the present or future color value (12) or corresponding property is predicted.

8. The method according to claim 7, wherein for the plurality of samples a fluorescent radiation (6) intensity (P) wavelength (λ) pair and/or a production process phase is or are used as further inputs; and/or that the at least one property of the fluorescent radiation (6) or present or future color value (12) or corresponding property of the solution (2) or product (3) is or are predicted.

9. The method according to claim 1, wherein a production process of a protein-containing solution (2) or of a protein-containing product (3) prepared therefrom is controlled based on the at least one property of the fluorescent radiation (6) or based on the future color value (12) or corresponding future property determined based on the fluorescent radiation (6).

10. The method according to claim 9, wherein at least one process parameter of the production process, in particular of a purifying step, is determined or controlled based on the at least one property of the fluorescent radiation (6) or future color value (12) or corresponding future property.

11. The method according to claim 1, wherein one or more of:
a fluorescence exciting radiation (5) has a maximum intensity (P) at a wavelength (λ) greater than 310 nm and less than 540 nm, preferably greater than 360 nm and less than 420 nm, in particular greater than 380 nm and less than 400 nm,
the fluorescent radiation (6) is detected within a wavelength (λ) range at least from 330 nm to 800 nm,
the fluorescent radiation (6) has a maximum intensity (P) at a wavelength (λ) greater than 330 nm and less than 800 nm, preferably greater than 420 nm and less than 600 nm, particularly greater than 450 nm and less than 530 nm, an intensity (P) maximum of the fluorescent radiation (6) is at a wavelength (λ) which is more than 60 nm and/or less than 130 nm above the wavelength (λ) at which the fluorescence exciting radiation (5) has a maximum intensity (P), and/or
the present or future color value (12) or corresponding property corresponds to a yellowish or brownish-yellowish coloration, preferably characterized in that light being transmittable, reflectable or scatterable by the solution (2) or product (3) with a maximum transmittance, reflectance or scattering at a wavelength (λ) greater than 560 nm and/or less than 620 nm.

12. The method according to claim 1, wherein the solution (2) or product (3) comprises recombinant proteins or antibodies, preferably monoclonal antibodies, and the present or future color value (12) or corresponding property is a characteristic of their, preferably pharmacological, usability.

13. The method according to claim 1, wherein a production process phase is taken into account for determining the present or future color value (12) or corresponding property.

14. The method according to claim 1, wherein a protein concentration is taken into account for determining the present or future color value (12) or corresponding property.

15. The method according to claim 1, wherein the solution (2) is a sample comprising a recombinant protein obtained in one of steps (i) to (vi) or (a) to (g) of a process comprising the steps of:
(i) cultivating a eukaryotic cell expressing the recombinant protein of interest in cell culture;
(ii) harvesting the recombinant protein;
(iii) purifying the recombinant protein, preferably comprising one or more of:
(a) affinity chromatography (AF)
(b) depth filtration (DF),
c) anion-exchange chromatography (AIEX)
(d) cation-exchange chromatography (CIEX),
(e) mixed-mode chromatography (MM),
(f) virus filtration (VF), and/or
(g) ultrafiltration/diafiltration (UF),
(iv) formulating the recombinant protein into a pharmaceutically acceptable formulation suitable for administration as product (3), in particular pure bulk drug substance in buffer solution (BDS),
wherein the fluorescent radiation (6) is excited with the sample, and wherein the future color value (12) or corresponding property is determined for a different, subsequent of the steps (i) to (iv) or (a) to (g).

16. The method according to claim 1, wherein the fluorescent radiation (6) has a wavelength (λ) and/or intensity (P), in particular a value pair of a wavelength (λ) and a corresponding intensity (P), based on which the present or future color value (12) or corresponding property of the solution (2) or product (3) is determined.

17. The method according to claim 1, wherein the fluorescent radiation (6) parameter is determined from a sample of the solution (2) or product (3) when held in a well (30) of a microtiter plate (29).

18. A method for producing a protein-containing solution (2) or a product (3) produced therefrom, comprising the steps of:
(i) cultivating a eukaryotic cell expressing a recombinant protein of interest in cell culture;
(ii) harvesting the recombinant protein;
(iii) purifying the recombinant protein; and
(iv) optionally formulating the recombinant protein into a pharmaceutically acceptable formulation suitable for administration; and
(v) obtaining at least one sample comprising the recombinant protein in steps (ii), (iii) and/or (iv);
wherein the sample is the solution (2) or product (3), and wherein the method further comprises performing a method of determining a future color value (12) or corresponding future property of a protein-containing solution (2) or of a protein-containing product (3) prepared therefrom, comprising:
exciting the fluorescent radiation (6) of the solution (2) or product (3),
measuring at least one property, preferably a spectrum (6A) or corresponding feature, of the fluorescent radiation (6), and
determining the future color value (12) or corresponding future property of the solution (2) or product (3) based on a correlation.

19. An arrangement (1) comprising a light source (4), a spectrometer (7) for measuring a fluorescent radiation (6), and a device (18) adapted to carry out a method of determining a future color value (12) or corresponding future property of a protein-containing solution (2) or of a protein-containing product (3) prepared therefrom, comprising:
exciting the fluorescent radiation (6) of the solution (2) or product (3),
measuring at least one property, preferably a spectrum (6A) or corresponding feature, of the fluorescent radiation (6), and
determining the future color value (12) or corresponding future property of the solution (2) or product (3) based on a correlation.
